(19) United States
(12) Patent Application Publication
(10) Pub. No.: US 2023/0017785 A1
(43) Pub. Date: Jan. 19, 2023

(54) COMPANION DIAGNOSTICS FOR COMPLEMENT INHIBITORS

(71) Applicant: THE JOHNS HOPKINS UNIVERSITY, Baltimore, MD (US)

(72) Inventors: Robert A. Brodsky, Brooklandville, MD (US); Xuan Yuan, Kingsville, MD (US)

(21) Appl. No.: 17/782,742

(22) PCT Filed: Dec. 7, 2020
(86) PCT No.: PCT/US2020/063621

§ 371 Date: Jun. 6, 2022

Related U.S. Application Data
(60) Provisional application No. 62/944,439, filed on Dec. 6, 2019.

(51) Int. Cl. G01N 33/573

(52) U.S. Cl. CPC G01N 33/573; G01N 2333/4716; G01N 2800/52; G01N 2333/96433

(57) ABSTRACT

The present invention relates generally to the field of disorders of complement activation. More specifically, the present invention provides methods and compositions useful for identifying patients having a complement-mediated disease that could benefit from treatment with complement inhibitors. As described herein, the present invention utilizes patient sera and flow cytometry in the companion diagnostic assay. More specifically, the present invention comprises evaluating complement activity via cell surface deposition of C5b-9 and, in further embodiments, complement-dependent cell killing (the modified Ham assay) using patient sera.

### CROSS REFERENCE TO RELATED APPLICATIONS

This application claims the benefit of U.S. Provisional Patent Application No. 62/944,439 filed on Dec. 6, 2019, which is incorporated by reference, herein, in its entirety.

### STATEMENT OF GOVERNMENTAL INTEREST

This invention was made with government support under HL133113, awarded by the National Institutes of Health. This government has certain rights in the invention.

### FIELD OF THE INVENTION

The present invention relates generally to the field of disorders of complement activation. More specifically, the present invention provides methods and compositions useful for identifying patients having a complement-mediated disease that could benefit from treatment with complement inhibitors.

### BACKGROUND OF THE INVENTION

The complement system is a key component of the innate immune system that provides host defense against a variety of pathogens including bacteria, fungi and viruses. Bains, A. C. and Brodsky, R. A., 31 BLOOD REVIEWS 213-223 (2017). Complement has diverse roles in a variety of cellular processes including bridging the innate and adaptive immune responses, maintaining homeostasis and preventing autoimmunity. Id.; Merle et al., 6 FRONT. IMMUNOL. 257 (2015); Merle et al., 6 FRONT. IMMUNOL. 262 (2015).

The complement cascade is a complex network of over 40 soluble and membrane proteins that can be activated by one of three primary pathways: (1) the lectin pathway; (2) the classical pathway; and (3) the alternative pathway. Id.; Ricklin et al., 11(9) NAT. IMMIUNOL. 785-97 (2010). Although the lectin, classical and alternative pathway are sometimes depicted as three separate pathways, they actually constitute different modes of activation for an interconnected downstream complement cascade. Id.; Oikonomopoulou et al., 34(1) SEMEN. IMMUNOPATHOL. 151-65 (2012).

Several promising therapeutic targets have been identified in the complement cascade, within each of the three pathways. A need exists for assay useful for identifying patients having complement-mediated diseases who could benefit from treatment with complement inhibitors.

### SUMMARY OF THE INVENTION

The present invention is based, at least in part, on the development of an assay to identify patients having a complement-mediated disease who could benefit from treatment with a complement inhibitor. As described herein, the present invention utilizes patient sera and flow cytometry in the companion diagnostic assay. More specifically, the present invention comprises evaluating complement activity via cell surface deposition of C5b-9 and, in further embodiments, complement-dependent cell killing (the modified Ham assay) using patient sera. In more specific embodiments, assessment of complement activation in patient serum comprises measuring deposition of terminal complement protein complexes (C5b-9) on PIGA null TF-1 cells following incubation with patient serum. The flow cytometry assays of the present invention can be combined with modified Ham assays to identify patients who can be treated with complement inhibitors. Inhibitors of the classical pathway, lectin pathway or the alternative pathway can be evaluated using the present invention.

Accordingly, in one aspect, the present invention provides method for identifying a patient having a complement-mediated disease as likely to benefit from treatment with a Factor D inhibitor. In one embodiment, the method comprises the steps of (a) in a first assay, (i) incubating serum obtained from the patient with a plurality of glycosylphosphatidylinositol-anchored protein (GPI-AP) deficient cells; (ii) staining the cells with an anti-β5b9 monoclonal antibody (mAb); and (iii) performing flow cytometry to measure C5b9 deposition on the cell membrane; (b) in a second assay, (i) incubating serum obtained from the patient with a plurality of GPI-AP deficient cells; (ii) adding a terminal complement inhibitor; (iii) staining the cells with an anti-β5b9 mAb; and (iv) performing flow cytometry to measure C5b9 deposition on the cell membrane; and (c) in a third assay, (i) incubating serum obtained from the patient with a plurality of GPI-AP deficient cells; (ii) adding the Factor D inhibitor; (iii) staining the cells with an anti-β5b9 mAb; and (iv) performing flow cytometry to measure C5b9 deposition on the cell membrane, wherein the patient is likely to benefit from treatment with the Factor D inhibitor if C5b9 deposition is blocked in the assays of steps (b) and (c), and wherein the patient is not likely to benefit from treatment with the Factor D inhibitor if C5b9 deposition is blocked in the assay of step (b) and not blocked in the assay of step (c).

In certain embodiments, the Factor D inhibitor comprises ACH-4471, ACH-5528 or ACHI-5548. In another aspect, the present invention provides a method of treating a patient having a complement-mediated disease comprising the step of administering a Factor D inhibitor to the patient identified using a method described herein.

In another aspect, the present invention provides a method for identifying a patient having a complement-mediated disease as likely to benefit from treatment with an inhibitor of the alternative pathway of complement. In one embodiment, the method comprises the steps of (a) in a first assay, (i) incubating serum obtained from the patient with a plurality of glycosylphosphatidylinositol-anchored protein (GPI-AP) deficient cells; (ii) staining the cells with an anti-β5b9 monoclonal antibody (mAb); and (iii) performing flow cytometry to measure C5b9 deposition on the cell membrane; (b) in a second assay, (i) incubating serum obtained from the patient with a plurality of GPI-AP deficient cells; (ii) adding a terminal complement inhibitor; (iii) staining the cells with an anti-β5b9 mAb; and (iv) performing flow cytometry to measure C5b9 deposition on the cell membrane; and (c) in a third assay, (i) incubating serum obtained from the patient with a plurality of GPI-AP deficient cells; (ii) adding the alternative pathway of complement inhibitor; (iii) staining the cells with an anti-C5b9 mAb; and (iv) performing flow cytometry to measure C5b9 deposition on the cell membrane, wherein the patient is likely to benefit from treatment with the alternative pathway of complement inhibitor if C5b9 deposition is blocked in the assays of steps (b) and (c), and wherein the patient is not likely to benefit from treatment with the alternative pathway of complement inhibitor if C5b9 deposition is blocked in the assay of step (b) and not blocked in the assay of step (c).

In particular embodiments, the alternative pathway of complement inhibitor comprises a Factor B inhibitor. In a specific embodiment, the Factor B inhibitor comprises IONIS-FB-LRx. In a further aspect, the present invention provides a method of treating a patient having a complement-mediated disease comprising the step of administering an alternative pathway of complement inhibitor to the patient identified using a method described herein.

In particular embodiments, the terminal complement inhibitor comprises a C5 inhibitor. In specific embodiments, the C5 inhibitor comprises eculizumab, ravulizumab, coversin, cemdisiran, LFG-316, SOBI005, SKY59, REGN3918, ABP959, GNR-45, zimura, RA101495, ISU305, or mubodina.

In a specific embodiment, the terminal complement inhibitor comprises an anti-β5 mAb. In a more specific embodiment, the anti-β5 mAb comprises eculizumab or ravulizumab.

In certain embodiments, the plurality of GPI-AP deficient cells is a Phosphatidylinositol glycan class A (PIGA) null cell line. In a specific embodiment, the plurality of GPI-AP deficient cells line is a cell line including, but not limited to, endothelial cells such as the TF-1 cell line. In another embodiment, the plurality of GPI-AP deficient cells is a PIGA null induced pluripotent stem cell line. In other embodiments, the plurality of GPI-AP deficient cells are obtained by biochemical treatment of cells to remove GPI-AP, for example, phosphatidylinositol-specific phospholipase C (PIPLC)-treated endothelial cells. In certain embodiments, the cell line is genetically or biochemically modified to remove complement regulatory proteins on the cell surface. For example, cell lines that are missing CD59 and/or CD55 that naturally protect cells from complement-mediated destruction could be used. Primary cells could also be used.

In certain embodiments, the COVID-19 patient is tested for mutations in a complement-related gene. In one embodiment, the mutation comprises a loss of function mutation in a complement inhibitory factor or a gain of function mutation of a complement activating factor. In a specific embodiment, the complement inhibitory factor comprises complement factor H (CFH), complement factor I (CFI), CD46, thrombomodulin (THBD, and complement receptor 1 (CR1). In another specific embodiment, the complement activating factor comprises complement factor B (CFB) and complement component C3.

### BRIEF DESCRIPTION OF THE FIGURES

FIG. 1A-1C. Complement activation in thrombotic APS and CAPS. Complement activation indicated by a positive mHam assay was detected in 35.6% of APS, and 85.7% of CAPS compared with only 6.8% of sera from patients with SLE (P<0.001) (FIG. 1A). Percentage (%) of patients with a positive mHam assay was also increased in a triple-positive aPL profile (positive for lupus anticoagulant, anti-β2-glycoprotein-1 Ab and anti-cardiolipin Ab) (FIG. 1B) and recurrent thrombosis (FIG. 1C).

FIG. 2A-2C. APS sera induce C5b-9 deposition. Flow cytometry demonstrated C5b-9 (membrane attack complex) deposition on the surface of PIGA null TF-1 cells in two patients (APS1 in FIG. 2A and APS2 in FIG. 2B). Sera from both patients led to C5b-9 deposition, which was completely blocked in the presence of eculizumab (anti-β5 monoclonal antibody). Adding factor D inhibitor (ACH4471) did not appreciably inhibit C5b-9 deposition in either patient which is also reflected in the mHam results shown in the top right of both panels. In the mHam, the dotted line at 20% non-viable cells indicates the threshold for a positive assay. FIG. 2C: aHUS patient PS-18. ACH-4471 completely inhibited C5b-9 deposition induced by sera from the patient. NS indicates normal serum, anti-β5 Ab indicates eculizumab, and FD inh indicates ACH4471 (factor D inhibitor). Stx1, Shiga toxin 1 (positive control); SSC, side scatter; anti-β5 Ab, eculizumab; ACH-4471, factor D inhibitor.

FIG. 3A-3B. Pathogenic anti-β₂GPI IgG induce C5b-9 deposition. FIG. 3A: Flow cytometry demonstrated that IgG anti-β₂GPI antibody from a patient with thrombotic APS (APS21) induced C5b-9 (membrane attack complex) deposition on the surface of PIGA null TF-1 cells. C5b-9 deposition was blocked completely with eculizumab and partially by a factor D inhibitor (ACH4471). FIG. 3B: IgM anti-β₂GPI from a patient with asymptomatic aPL (APS 3) led to a very small increase in C5b-9 deposition. NS indicates normal serum, NS(H) indicates heat inactivated normal serum, anti-β5 Ab indicates eculizumab, and FD inh indicates ACH4471 (factor D inhibitor).

FIG. 4A-4B. CAPS sera activate complement and induce C5b-9 deposition. FIG. 4A: Sera from a patient with CAPS (CAPS5) induced C5b-9 deposition on PIGA null TF-1 cells. C5b-9 deposition was blocked by eculizumab (C5 inhibitor) and partially by a factor D inhibitor (ACH4471). The mHam assay also showed positive tests (>20% non-viable cells) with CAPS sera, which was attenuated by eculizumab and a factor D inhibitor. The patient had a CR1 V2125L variant. FIG. 4B: NS indicates normal serum, NS(H) indicates heat inactivated normal serum, anti-β5 Ab indicates eculizumab, and FD inh indicates ACH4471 (factor D inhibitor).

FIG. 5. Germline variants identified in patients with CAPS. Single nucleotide variant location is provided, along with a schematic diagram of CFHR3-CFHR1 deletion (identified in 3 patients). Exons are indicated as blocks. Short consensus repeats (SCRs) are numbered for both CR1 (C3b/C4b receptor) and CFHR4, with the most common isoforms shown. Protein domains include LHR, long homologous repeat; TM, transmembrane domain; CR, cytoplasmic region; CLEC, C-type lectin domain; TIME, thrombomodulin EGF-like domain. Regulatory regions include SA, sialic acids; C3b thioester-containing domain (TED); MBL, mannose binding lectin: CA, cofactor activity; DAA, decayed accelerating activity; GAG, glycosaminoglycans. Binding sites for calcium, integrins and complement proteins are shown.

FIG. 6. Proposed ‘two-hit’ model for CAPS. The present inventors propose a pathogenic model in which aPL induce complement activation and cause thrombosis.

Patients who also have a pathogenic “loss of function” mutation in a complement inhibitory factor (CFH, CFI, CD46 (MCP), THBD, CR1) or a “gain of function” mutation of a complement activating factor (CFB, C3) are likely to be predisposed to uncontrolled complement activation, which could lead to disseminated thrombosis and ischemic multi-organ failure in the setting of a complement amplifying trigger such as infection, surgery, or autoimmune disease.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

The compositions and methods described herein are useful for evaluating complement inhibitors, for example, factor D inhibitors, and identifying patients that would benefit from such treatment. The present invention can be used to treat patients having a disorder that is mediated by the complement pathway, and in particular embodiments, a pathway that is modulated by complement factor D. In certain embodiments, the disorder is an inflammatory disorder, an immune disorder, or an autoimmune disorder. In one embodiment, the disorder is an ocular disorder. Complement-mediated diseases that may be treated or prevented include, but are not limited to, inflammatory effects of sepsis, systemic inflammatory response syndrome (SIRS), ischemia/reperfusion injury (I/R injury), psoriasis, myasthenia gravis, system lupus erythematosus (SLE), paroxysmal nocturnal hemoglobinuria (PNH), hereditary angioedema, multiple sclerosis, trauma, burn injury, capillary leak syndrome, obesity, diabetes, Alzheimer's dementia, stroke, schizophrenia, epilepsy, age-related macular degeneration, glaucoma, diabetic retinopathy, asthma, allergy, acute respiratory distress syndrome (ARDS), atypical hemolytic uremic syndrome (aHUS), hemolytic uremic syndrome (HUS), cystic fibrosis, myocardial infarction, lupus nephritides, Crohn's disease, rheumatoid arthritis, atherosclerosis, transplant rejection, prevention of fetal loss, biomaterial reactions (e.g. in hemodialysis, implants), C3 glomerulonephritis, abdominal aortic aneurysm, neuromyelitis optica (NMO), vasculitis, neurological disorders, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during I L-2 therapy, inflammatory disorders, inflammation of autoimmune diseases, adult respiratory distress syndrome, thermal injury including burns or frostbite, myocarditis, post-ischemic reperfusion conditions, balloon angioplasty, post-pump syndrome in cardiopulmonary bypass or renal bypass, hemodialysis, renal ischemia, mesenteric artery reperfusion after aortic reconstruction, immune complex disorders and autoimmune diseases, SLE nephritis, proliferative nephritis, liver fibrosis, hemolytic anemia, tissue regeneration and neural regeneration.

In addition, other known complement related disease are lung disease and disorders such as dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome, pulmonary vasculitis, Pauci-immune vasculitis, immune complex-associated inflammation, uveitis (including Behcet's disease and other sub-types of uveitis), antiphospholipid syndrome, arthritis, autoimmune heart disease, inflammatory bowel disease, ischemia-reperfusion injuries, Barraquer-Simons Syndrome, hemodialysis, systemic lupus, lupus erythematosus, transplantation, diseases of the central nervous system and other neurodegenerative conditions, glomerulonephritis (including membrane proliferative glomerulonephritis), blistering cutaneous diseases (including bullous pemphigoid, pemphigus, and epidermolysis bullosa), ocular cicatrical pemphigoid, MPGN II, uveitis, adult macular degeneration, diabetic retinopathy, retinitis pigmentosa, macular edema, Behcet's uveitis, multifocal choroiditis, Vogt-Koyangi-Harada syndrome, imtermediate uveitis, birdshot retino-chorioditis, sympathetic ophthalmia, ocular dicatricial pemphigoid, ocular pemphigus, nonarteric ischemic optic neuropathy, postoperative inflammation, and retinal vein occlusion.

In some embodiments, complement mediated diseases include ophthalmic diseases (including early or neovascular age-related macular degeneration and geographic atrophy), autoimmune diseases (including arthritis, rheumatoid arthritis), respiratory diseases, cardiovascular diseases. In other embodiments, complement mediated diseases include diseases and disorders associated with fatty acid metabolism, including obesity and other metabolic disorders.

The compositions and methods of the present invention can be used to assess whether a patient can benefit from a complement inhibitor. Examples of complement inhibitors generally include a protease inhibitor, a soluble complement regulator, a therapeutic antibody (monoclonal or polyclonal), complement component inhibitors, receptor agonists, or siRNAs.

Non-limiting examples of active agents in these categories include:

Protease inhibitors: plasma-derived C1-INH concentrates, for example CETOR® (Sanquin), BERINERT® (CSL Behring, Lev Pharma), and CINRYZE®; and recombinant human C1-inhibitors, for example RHUCIN® and RUCONEST® (Pharming);

Soluble complement regulators: Soluble complement receptor 1 (TP10) (Avant Immunotherapeutics); sCR1-sLe^{X}/TP-20 (Avant Immunotherapeutics); MLN-2222/CAB-2 (Millenium Pharmaceuticals); Mirococept (Inflazyme Pharmaceuticals);

Therapeutic antibodies: Eculizumab/Soliris (Alexion Pharmaceuticals); Pexelizumab (Alexion Pharmaceuticals); Ofatumumab (Genmab A/S); TNX-234 (Tanox); TNX-558 (Tanox); TA106 (Taligen Therapeutics); Neutrazumab (G2 Therapies); Anti-properdin (Novelmed Therapeutics); HuMax-CD38 (Genmab A/S);

Complement component inhibitors: Compstatin/POT-4 (Potentia Pharmaceuticals); ARC 1905 (Archemix);

Receptor agonists: PMX-53 (Peptech Ltd.); JPE-137 (Jerini); JSM-7717 (Jerini);

Others: Recombinant human MBL (rhMBL; Enzon Pharmaceuticals).

Inhibitors that can be evaluated using the compositions and methods of the present invention include, but are not limited to, OMS721 (OMS 00620646) (Omeros); Ravulizumab (ALXN1210) (Alexion); Coversin (Nomacopan) (Akari Therapeutics); CCX168 (Avacopan) (ChemoCentryx); IFX1 (CaCP29 (InfaRx); AMY-101 (Amyndas); APL-2 (Apellis); LNP023 (Novartis); Cemdisiran (ALN-CC5) (Alnylam); ClINH (Berinert) (CSL Behring); LFG-316 (Novartis).

In particular embodiments, the present invention can be used to identify which patients would benefit from a C5 inhibitor including, but not limited to, Eculizumab (Alexion); Ravulizumab (Alexion); Coversin (Akari); Cemdisiran (Alnylam); LFG-316 (Novartis); SOBI005 (Sobi); SKY59 (RG6107/R07112689 (Chugai and Roche)); REGN3918 (Regeneron); ABP959 (Amgen); GNR-45 (Generium); Zimura (Ophthotech); RA101495 (Ra Pharma); ISU305 (ISU ABXIS); and Mubodina (Adienne). In other embodiments, a C5a inhibitor can include IFX-1 (InflaRx) and Avacopan (CCX168 (Chemocentryx)). In further embodiments, a C5aR1 inhibitor can include ALS-205 (Alsonex); DF2593A (Dompe); and IPH5401 (Innate Pharma). In other embodiments, a C6 inhibitor can be evaluated including, but not limited to, Regenmab (Regenesance) and C6-LNA (Regenesance).

In particular embodiments, a C3 inhibitor can be evaluated including, but not limited to, AMY-101 (Amyndas); APL-1 (Apellis); APL-2 (Apellis); and APL-9 (Apellis).

A Factor B inhibitor includes IONIS-FB-LRx (Ionis, GSK). Other Factor B inhibitors include LNP203 (Novartis (Basel, Switzerland); Schubert et al., 116(16) PROC. NATL. ACAD. SCI. USA 7926-31 (2019)), anti-FB SiRNA (Alnylam Pharmaceuticals, Cambridge, Mass.); TA106 (monoclonal antibody, Alexion Pharmaceuticals, New Haven, Conn.); SOMAmers (aptamers, SomaLogic, Boulder, Colo.); bikaciomab (Novelmed Therapeutics, Cleveland, Ohio); complin (see, Kadam et al., J. Immunol. 2010, DOI: https://doi.org/10.4049/jimmunol.1000200).

In certain embodiments, a Factor D inhibitor can be evaluated including ACH-4471 (Achillion), ACH-5528 (Achillion) and ACHI-5548 (Achillion). Other Factor D inhibitors from Achillion include those described in U.S. Pat. No. 10,464,956 (compounds claimed in claims 1-12); U.S. Pat. No. 10,428,095 (compounds claimed in claims 1-2); U.S. Pat. No. 10,428,094 (compounds claimed in claims 1-28); U.S. Pat. No. 10,385,097 (compounds described in claims 1 and 20-27); U.S. Pat. No. 10,370,394 (compounds described in claims 1 and 13-35); U.S. Pat. No. 10,301,336 (compounds described in claims 1 and 3); U.S. Pat. No. 10,287,301 (compounds claimed in claims 1-3); U.S. Pat. Nos. 10,253,053; 10,189,869 (compounds described in claims 1 and 12-36); U.S. Pat. No. 10,138,225 (compounds described in claims 1 and 2-24); U.S. Pat. No. 10,106,563 (compounds claimed in claims 1-20); U.S. Pat. No. 10,100,072 (compounds claimed in claims 1-50); U.S. Pat. No. 10,092,584 (compounds described in claims 1, 15-31 and 38-41); U.S. Pat. No. 10,087,203 (compounds claimed in claims 1-21); U.S. Pat. No. 10,081,645 (compounds claimed in claims 1-40); U.S. Pat. No. 10,011,612 (compounds claimed in claims 1-3); U.S. Pat. No. 10,005,802 (compounds claimed in claims 1-38); U.S. Pat. No. 10,000,516 (compounds described in claims 1 and 13-20); U.S. Pat. No. 9,828,396 (compounds claimed in claims 1 and 12-40); U.S. Pat. No. 9,796,741 (compounds claimed in claims 1 and 12-48); U.S. Pat. No. 9,758,537 (compounds claimed in claims 1-39); U.S. Pat. No. 9,732,104 (compounds claimed in claims 1-68); U.S. Pat. No. 9,732,103 (compounds claimed in claims 1-29); U.S. Pat. No. 9,695,205 (compounds claimed in claims 1-56); U.S. Pat. No. 9,663,543 (compounds claimed in claims 1-49); U.S. Pat. No. 9,643,986 (compounds claimed in claims 1-24); and U.S. Pat. No. 9,598,446 (compounds claimed in claims 1-7). Another Factor D inhibitor includes Lampalizumab (Genentech).

The compositions and methods of the present invention can be used to assess inhibitors of the classical pathway of complement including, but not limited to, Clq inhibitors (ANX005, ANX007 (Annexon)); Cls inhibitors (BIVV020 (Bioverativ)); C2 inhibitors (PRO-02 (Broteio/Argen-x)), as well as inhibitors of the lectin pathway including, but not limited to, MASP3 inhibitors (OMS906 (Omeros)).

The present invention also comprises testing a patient for mutations in complement related genes, specifically, for mutations in genes that inhibit regulation of APC or mutations that directly activate APC. Patients can be tested for mutations in complement factor H (CFH), CFH-related proteins (CFHR1, CFHR2, CFHR3, CFHR4, CFHR5), complement factor I (CFI), CD46 (membrane cofactor protein, MCP), complement factor B (CFB), complement component C3 (C3), thrombomodulin (THBD), plasminogen, diacylglycerolkinase-E (DGKE), complement factor D (CFD), and complement receptor 1 (CR1).

In more particular embodiments, patients are tested for loss of function mutation(s) in a complement inhibitory factor (CFH, CFI, CD46 (MCP), THBD, CR1) or a gain of function mutation(s) of a complement activating factor (CFB, C3). Such mutations are known in the art. These patients are likely to be predisposed to uncontrolled complement activation, which could lead to disseminated thrombosis and multi-organ failure in the setting of a complement amplifying trigger such as infection (COVID-10), as well as surgery, pregnancy or autoimmune disease.

In some embodiments, depending on the mutation(s), a COVID-10 patient may benefit from a terminal complement inhibitor (e.g., anti-β5 antibody (eculizumab)) or an APC inhibitor such as a Factor D inhibitor (e.g., ACH-4471) or a Factor B inhibitor (IONIS-FB-LRx), as well as administration of Factor H.

The compositions and methods of the present invention utilize glycosylphosphatidylinositol-anchored protein (GPI-AP) deficient cells. In particular embodiments, cells are biochemically treated to remove GPI-AP. In other embodiments, the plurality of GPI-AP deficient cells is a phosphatidylinositol glycan class A (PIGA) null mutant cell line. The present inventors previously established a PIGA mutant cell line derived from TF1 cells. PIGA is a gene required for the first step in the biosynthesis of glycosylphosphatidylinositol (GPI), a lipid moiety that anchors dozens of proteins to the cell surface. Two of the GPI-anchored proteins that are defective in the TF1 cell line are CD55 and CD59. The proteins both regulate complement. CD55 blocks C3 convertases and CD59 interferes with/blocks terminal complement activation.

In particular embodiments, the compositions and methods of the present invention use this cell line as a reporter cell line for activation of complement in patient serum. In certain embodiments, a flow cytometry assay is performed as described herein. In other embodiments, a modified Ham assay is performed. Briefly, about 5 cc of serum is collected from patients, diluted 1:4 with growth medium and viability of the PIGA mutant TF1 cells is measured after 30 minutes using a WST1 assay. To confirm that the cell kill is associated with complement, the cells are stained with a monoclonal antibody to C5b9 (terminal complement attack) and assay the staining by flow cytometry.

In a non-limiting embodiment, the modified Ham assay may be conducted as follows:

Blood is collected in serum separation tubes and is immediately centrifuged at 4° C. Serum is separated and stored at −80° C. Heat inactivation is performed the same day of the experiment, incubating the serum at 56° C. for 30 minutes.

The cell viability assay is performed on a glycosylphosphatidylinositol-anchored proteins (GPI-AP) deficient TF-1 cell line that has been previously established. See Savage et al., 37(1) EXP. HEMATOL. 42-51 (2009). Cells are maintained in RPMI 1640 medium supplemented with 2 ng/mL GM-CSF, 2 mM 1-glutamine, penicillin/streptomycin, and 10% fetal calf serum under BL2 lab containment.

Cells are plated in a U-shaped 96-well plate at a density of approximately 4.000 cells/well and cultured until confluent. Then, cells are washed with PBS and incubated with serum at a concentration of 1:4 for 30 minutes at 37° C. Serum is diluted in GVB (gelatin veronal buffer, Sigma). Cells are washed again with PBS and incubated with the cell proliferation reagent (4[3-(4-lodophenyl)-2-(4-nitrophenyl)-2H-5-tetrazolio]-1.3-benzene disulfonate/WST-1, Roche) for 3 hours at 37° C. Wst-1 is diluted in the cell culture medium at a concentration of 1:10 and 100 μl of Wst-1 solution is added per well. Absorbance is measured in a microplate (ELISA) reader at 450 nm with a reference wavelength at 650 nm, according to the manufacturer's instructions and previous publication. See Taylor et al., 23(4) PEDS 251-60 (2010). The colorimetric assay is based on cleavage of the tetrazolium salt, WST-1, by mitochondrial dehydrogenases in viable cells.

In certain embodiments, absorbance values of each sample are normalized after subtraction of the absorbance value of a blank cell. Percentage of viable cells is expressed as a ratio of the absorbance of each sample multiplied by 100, to the absorbance of the same sample's heat-inactivated control. Percentage of dead cells is calculated after subtracting percentage of viable cells from 100.

The cell viability indicator can be any substance, composition or compound capable of providing a particular change which selectively identifies the presence of viable cells in the biological sample. In particular embodiments, the cell viability indicator is a tetrazole. Tetrazoles serve as a substrate for an enzymatic reaction, which provides a colorimetric measure of the activity of cellular metabolic enzymes that reduce the tetrazoles to formazan. Such tetrazoles include, but are not limited to, 3-(4,5-Dimethylthiazol-2-yl)-2.5-diphenyltetrazolium bromide (MTT), 2,3-bis-(2-methoxy-4-nitro-5-sulfophenyl)-2H-tetrazolium-5-carboxanilide (XTT), 3-(4,5-dimethylthiazol-2-yl)-5-(3-carboxymethoxyphenyl)-2-(4-sulfo-phenyl)-2H-tetrazolium (MTS) or Water soluble Tetrazolium salts (WTSs), for example WST-1 (2-(4-Iodophenyl)-3-(4-nitrophenyl)-5-(2,4-disulfophenyl)-2H-tetraz-olium) and WST-8 (2-(2-methoxy-4-nitrophenyl)-3-(4-nitrophenyl)-5-(2,4-disulfophenyl)-2H-tetrazolium).

Other suitable cell viability indicator reagents may also be used. In certain embodiments of the invention, biological samples are exposed to fluorescent dyes to provide information regarding the biological function of the cells within the sample. Such fluorescent dyes include “live cell” dyes (e.g., calcein AM) which selectively accumulate within viable cells and which are modified within the environment of viable cells to produce fluorescent chemical species. Such “live cell” dyes selectively render viable cells fluorescent whilst leaving non-viable cells unstained. Variants of these “live cell” dyes have chemical groups such that they become covalently attached to cellular proteins during fixation so that the dye is retained within the cell for prolonged periods of time. Other fluorescent dyes include “dead cell” dyes (e.g., propidium iodide or ethidium bromide homodimer) which can enter and stain non-viable cells but which are excluded from viable cells.

In further embodiments, assays that are based on the incorporation of labeled nucleotide or nucleotide analogs into the DNA of cells can be used. In such assays, cells are exposed to a labeled nucleotide, e.g., ¹⁴C-thymidine, 3H-thymidine, or 5-bromo-2-deoxyuridine (BrdU). Proliferation is quantified by measuring the amount of labeled nucleotide taken up by the cells. Radiolabeled nucleotides can be measured by radiodetection methods; antibodies can be used to detect incorporation of BrdU.

Still other assays measure cellular viability/proliferation as a function of ATP production. For example, the luciferase enzyme catalyzes a bioluminescent reaction using the substrate luciferin. The amount of bioluminescence produced by a sample of cells measures the amount of ATP present in the sample, which is an indicator of the number of cells.

In specific embodiments, the assay is repeated using complement inhibitors and noting its effect on cell viability. In other embodiments, flow cytometry is used to measure C5b9 deposition on cell membranes.

The present invention also provides kits for performing the assays described herein. In particular embodiments, the kit comprises a GPI-AP deficient cell line. In another embodiment, the kit can also comprise growth media for the cell line. The kit can further comprise a substrate or support for containing the cells. In other embodiments, the kit comprises a positive and negative control. The kit can also comprise the necessary buffers for preparing, washing, etc. of the samples and/or cells. In a specific embodiment, the kit also comprises the components for conducting the cell viability assay including the cell proliferation reagent (e.g., WST-1), cell viability indicator reagent and the like. In other embodiments, the kit comprises the components for conducting flow cytometry to measure C5b9 deposition on cell membranes. For example; the kit can comprise anti-β5b9 antibody. The kit can further comprise secondary antibody and labels (which could be conjugated to the primary and/or secondary antibodies). In another embodiment, the kit can comprise anti-C3c antibodies.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Antiphospholipid syndrome (APS) is an acquired thrombophilia characterized by thrombosis affecting the venous or arterial vascular systems and/or obstetrical morbidity with the persistent presence of antiphospholipid antibodies (aPL), including lupus anticoagulant (LA), anticardiolipin antibody (aCL), and anti-beta-2-glycoprotein-I (β2GPI).¹ Anti-β2GPI antibodies are considered the primary pathogenic antibody in APS.²⁻⁴ The mechanisms by which aPL induce thrombosis are unclear. Multiple mechanisms have been proposed including inhibition of the natural anticoagulant and fibrinolytic systems,⁵⁻⁸ activation of vascular cells including endothelial cells,⁹ platelets¹⁰ and monocytes,¹¹ procoagulant effects of extracellular vesicles,¹² disruption of the annexin A5 shield on cellular surfaces,¹³ and complement activation.¹⁴⁻²⁰ The lack of a unifying mechanism likely reflects heterogeneity in pathogenic antibodies and disease biology.

Distinguishing benign from pathogenic aPL is a major gap in APS care and research. The presence of a LA^{21,22} and triple-positivity (presence of LA, aCL and anti-beta-2-glycoprotein-I antibody)^{23,24} are strong predictors of thrombotic risk in APS at a population but not individual, level. Predicting which patients with aPL are at risk of a first or subsequent thrombotic event remains challenging.²⁵ Long-term anticoagulation with a vitamin K antagonist remains the standard of care for thrombotic APS.²⁶ A severe form of APS characterized by widespread thrombosis and multi-organ failure developing over less than a week, termed catastrophic APS (CAPS), affects a subset (˜1%) of APS patients. CAPS often presents as a thrombotic microangiopathy and has a fulminant course with >40% mortality despite best available therapy^{26,27} indicating a need for therapies beyond anticoagulation.²⁶

Complement inhibition has emerged as an attractive therapeutic strategy based on evidence of complement activity in patients with APS, murine models that indicate a critical role of complement in aPL-mediated thrombosis¹⁴⁻¹⁷ and obstetric¹⁸⁻²⁰ complications, and reports of the efficacy of terminal complement inhibition with eculizumab in patients with refractory thrombotic APS²⁸ and CAPS.²⁹⁻³² Increased complement activation products including C5b-9,³³ fragment Bb, and C3a^{34,35} have been observed in sera of patients with APS; however, the association of APS-related thrombosis with serologic evidence of complement activation is inconsistent. 35,36

In this prospective study, the present inventors investigated complement activity via cell surface deposition of C5b-9 and complement-dependent cell killing (the modified Ham assay) using patient sera and show that complement activation is associated with thrombotic events in APS. In addition, we demonstrate complement activation by patient-derived purified anti-β2GPI in vitro. Finally, targeted sequencing was performed to test the hypothesis that CAPS is associated with rare germline variants in complement regulatory genes, serving as a ‘second hit’ that accounts for the more severe phenotype observed in this disease.

### Materials and Methods

Patients and samples. Between January 2015 and June 2019, the present inventors prospectively recruited patients with thrombotic APS, CAPS, and SLE from the Johns Hopkins Complement Associated Disorders Registry and the Hopkins Lupus Cohort.³⁷ Patients with CAPS were also recruited from the hematology services at the Cleveland Clinic, Cleveland, Ohio and McMaster University, Hamilton, ON. Patients were diagnosed with thrombotic APS based on International Society on Thrombosis and Hemostasis (ISTH) criteria including one or more clinical episodes of arterial, venous, or small vessel thrombosis, and the presence of LA, aCL antibody of the IgG/IgM isotype, or anti-β2GPI antibody of the IgG/IgM isotype detected on at least two occasions at least 12 weeks apart.¹ Patients were classified as single-, double-, or triple-positive based on positive assays for one, two or all three of LA, anti-β₂GPI antibodies, and aCL antibodies. Patients with recurrent thrombosis confirmed by imaging at any time prior to enrollment or during follow-up in the registry were classified as having recurrent thrombosis. For patients with multiple studies for aPL, we used the tests drawn closest to the study sample. CAPS was diagnosed according to international consensus criteria including involvement of three or more organs, development of manifestations within a period of a week, histologic confirmation of small vessel thrombosis, and laboratory confirmation of the presence of aPL.³⁸ The diagnosis of definite CAPS requires all four criteria, while probable CAPS is diagnosed if three criteria are met (but tissue biopsy is not obtained, laboratory testing cannot be repeated due to death, or multi-organ thrombosis develops over more than a week but less than a month, despite anticoagulation).³⁸ The present inventors included patients with both definite and probable CAPS because biopsies to confirm confirmation of small vessel thrombosis are commonly omitted in critically ill patients who otherwise meet criteria for CAPS, and outcomes of patients with probable CAPS are comparable to patients with definite CAPS.^{39,40} SLE was diagnosed according to the Systemic Lupus International Collaborating Clinics Criteria (SLICC).⁴¹

For genetic analysis, we used DNA samples from healthy pregnant women recruited from the obstetrics clinics at Johns Hopkins Hospital as a control cohort as well as patients with atypical hemolytic uremic syndrome (aHUS) as a reference cohort for validation of our custom sequencing panel. aHUS was diagnosed if their first manifestation of the syndrome met the following criteria: (1) platelet count <100×10⁹/L, (2) serum creatinine >2.25 mg/dL, and (3) ADAMTS13 activity >10%. These criteria have been used in previous studies to clinically differentiate patients with aHUS.⁴² Blood was collected by venipuncture in serum separation tubes and was immediately centrifuged at 4° C. Serum was separated and stored at −80° C. Whole blood was used to generate genomic DNA for targeted gene sequencing using a Qiagen DNeasy Blood & Tissue Kit. All samples and data were de-identified and coded after collection. Samples were obtained either from patients enrolled into existing registries, or were submitted by clinical centers as part of diagnostic testing. This study was approved by the institutional review board at Johns Hopkins University.

Patient derived anti-β₂GPI antibodies. Anti-β₂GPI antibodies from two patients were affinity purified using a column of Affigel HZ to which purified human β₂GPI was coupled (Bio-Rad Laboratories, Hercules, Calif.), as previously described.⁴³ IgG purity was assessed by reduced SDS-PAGE. Complement activation (C5b-9 deposition and complement dependent cell killing in mHam) induced by patient-derived anti-β₂GPI antibodies was tested by adding patient-derived anti-β₂GPI antibodies to normal human serum (Cat. NHS, Complement Technology, Inc.).

Modified Ham assay. The mHam assay has been validated to assess complement activation in patient serum and was performed as described previously.^{42,44} Briefly, PIGA null TF-1 cells were plated in 96-well plates at a density of 6700 cells/well and cultured until confluent. Briefly, PIGA null TF-1 cells were plated in 96-well plates in gelatin veronal buffer with Ca and Mg (GVB⁺⁺) (Cat. B102, Complement Technology, Inc.) at a density of 6,700 cells/well. Test serum (20 μL at 1:5 dilution) was added to cells and incubated at 37° C. for 45 minutes with constant shaking. After incubation, cells were again washed with phosphate buffered saline and incubated with the cell proliferation reagent, 4-[3-(4-lodophenyl)-2-(4-nitrophenyl)-2H-5-tetrazolio]-1.3-benzene disulfonate/WST-1 (Roche, Switzerland) at dilution 1:10 for 2 hours at 37° C. Absorbance was measured in a plate reader (ELX808, BioTeK, Winooski, Vt.) at 450 nm with a reference wave length at 630 nm. The colorimetric assay is based on cleavage of the tetrazolium salt, WST-1, by mitochondrial dehydrogenases in viable cells. The percentage of live cells was calculated as the ratio of absorbance of the sample to its heat inactivated control multiplied by 100. The percentage of non-viable cells (100−percentage viable cells) is a measure of complement activation. Based on prior experiments, 20% non-viable cells (cell killing) is established as the threshold for a positive test.^{42,44} All assays were performed in triplicate for replication.

As a positive control for the mHam, Shiga toxin⁴⁵ (Cat. SML0562, Sigma-Aldrich, St. Louis, Mo.) 10 μg/mL was added to NHS and incubated at 37° C. for 15 minutes, followed by the addition of the cells. Heat-inactivated serum was used an internal negative control for each sample. To confirm complement dependence of cell killing, the mHam was performed after adding an anti-β5 monoclonal antibody (10 μg) (Alexion pharmaceuticals). The assays were also performed with a small molecule factor D inhibitor⁴⁶ (0.33 μM) (ACH-4471, Achillion Pharmaceuticals) to evaluate the contribution of the alternative versus classical/lectin pathways.

Flow cytometry for C5b-9 deposition. Flow cytometry was performed to evaluate cell surface deposition of C5b-9 deposition on PIGA null TF1 cells. Cells were seeded in V-bottom 96-well plates (1.2×10⁵ cells/well) either in GVB⁰.MgEGTA (pH 6.4) buffer for alternative pathway activation or GVB++ (pH 7.4) buffer for classical pathway activation followed by addition of APS patient serum (or patient-derived aPL added to normal human serum). The reaction was incubated for 15 min at 37° C., and stopped by adding FACS buffer/EDTA. Cell pellets were separated by centrifugation at 1900 rpm for 3 minutes at room temperature and then stained with Alexa 488 conjugated anti-C3c antibody (Cat. 4212, Abcam, dilution at 1:100) and anti-β5b-9 monoclonal primary antibody (Cat. 66768, Abcam, dilution at 1:100), followed by Alexa 647 conjugated secondary antibody. C5b-9 deposition was measured by a BD FACSCalibur. Ten thousand events were recorded per sample and analyzed using FlowJo software version 10.5.3 (FlowJo Inc). Normal human serum was used as a negative control for flow cytometry assays. We also evaluated the effect of adding anti-C5 mAb and ACH-4471 on C5b-9 deposition.

Targeted sequencing. Genomic DNA was quantitated using Qiagen Qubit fluorometric assay and 50 ng of DNA was used as input for targeted sequencing with a custom Ampliseq panel (Illumina). Amplicons were designed to cover all exons of 15 genes with known function related to complement activation/regulation (CFH, CFB, CFI, CFD, CFP, CFHR1, CFHR2, CFHR3, CFHR4, CFHR5, C3, CD46 (MCP), THBD, CR1, DGKE) with amplicon length between 100-350 base pairs (bp) (median 307 bp, mean 285 bp). Libraries were generated per manufacturer's protocol. Briefly, targets were amplified using a Veriti 96-well Thermal Cycler, followed by amplicon digestion, index ligation and purification. Amplicons were then amplified and purified, followed by quantification via Qubit fluorometric assay. Library quality was assessed using Agilent 2100 Bioanalyzer. Subsequently, libraries were normalized and pooled prior to sequencing via Illumina MiSeq using v3 (600-cycle) reagents performed by the Genetic Resources Core Facility at Johns Hopkins School of Medicine. MiSeq optimization and quality control was performed by the GRCF, and mean amplicon coverage for all samples was 640×. Analysis of raw sequencing data (FASTQ) was performed using the DNA Amplicon pipeline (v2.1.1) via the Illumina BaseSpace platform. Alignment to (GRCh37/hg19) human genome reference was performed using the banded Smith-Waterman algorithm in the targeted regions. Variant calls were made using an Illumina-developed germline variant caller and filtered using VariantStudio software (v3.0). Variants not passing Illumina's variant quality filters were excluded, followed by filtering using the following criteria to identify rare germline single nucleotide variants and indels: 1) depth greater than 50×; 2) non-synonymous coding region or splice variants; 3) variant allele frequency between 40% and 60%; 4) minor allele frequency less than 0.005 in any ethnic population in the genome aggregation database (gnomAD, total 141,456 individuals). Large deletions were determined by complete loss of signal for multiple consecutive amplicons. Homozygous deletion of CFHR1 and CFHR3, reported to occur in approximately 2% of the population, was included in our analysis due to its association with CFH antibody formation and association with aHUS.⁴⁷

Data analysis. Data were summarized as counts (percent) and medians (Q25%-75%) or mean (+/−standard deviation) for categorical and continuous variables, respectively. The chi-squared test was used to compare the proportion of patients with positive mHam in the following groups: (i) SLE, APS and CAPS, (ii) single, double and triple-positive APS, and (iii) patients with and without a history of thrombosis. The paired t-test was used to compare C5b-9 deposition in patient sera with their own heat inactivated negative controls. The chi-squared test was used to compare the rate of rare germline variants in complement genes in different groups. P<0.05 was considered significant for all analyses. STATA ver. 23 (STATA Corp) was used to perform analysis.

### Results

Patient characteristics. Between 2015 and 2019, we enrolled 59 patients with thrombotic APS, 74 patients with SLE, and 10 patients with CAPS. Among APS patients, 22 (37.3%) were single positive, 15 (25.4%) were double positive, and 22 (37.3%) were triple-positive. Among APS, 37 had had venous thrombosis, 13 had had arterial thrombosis and 9 had had both venous and arterial thrombotic events. Recurrent thrombotic events occurred in 37.3% (22 of 59) APS patients, of which 14 developed recurrent thrombosis on therapeutically dosed anticoagulation (including three with recurrent thrombosis while on a direct oral anticoagulant).

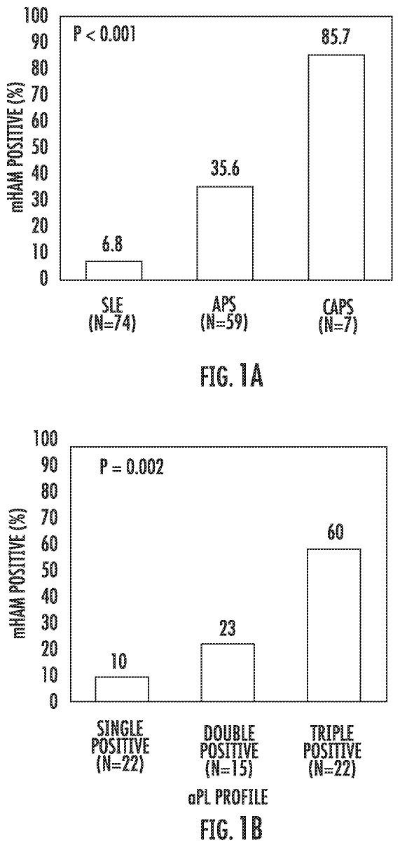

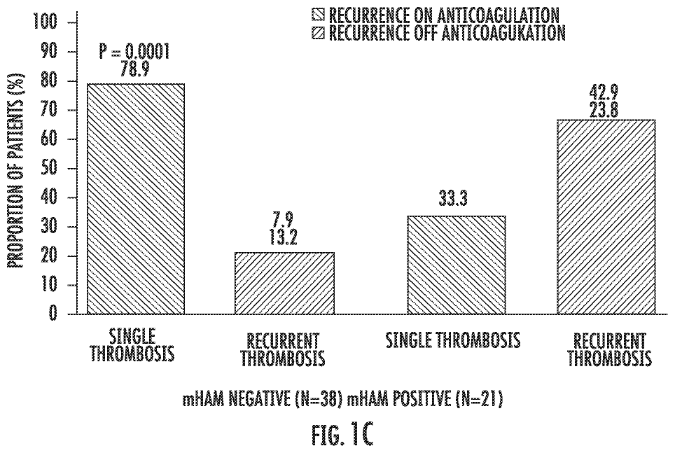

Thrombotic APS is associated with complement activation. Complement activity was assessed via complement-mediated killing of nucleated cells measured using the mHam assay, which has been previously validated in both disease states and normal subjects. A positive mHam assay (>20% cell killing)^{42,44,48} was detected in 35.6% (21 of 59) patients with thrombotic APS and 85.7% (6 of 7 with available sera) of CAPS compared with 6.8% (5 of 74) with SLE, (P<0.001) (FIG. 1A).

Among patients with APS, mHam positivity was associated with triple-positivity (60%), over double (23%) or single positivity (10%) (P=0.002) (FIG. 1B). mHam positivity was also associated with recurrent thrombosis in 66.7% patients (42.9% recurrence on anticoagulation and 23.8% recurrence off anticoagulation) than a single event (33.2%). APS patients with a negative mHam were more likely to have had a single thrombotic event (78.9%) than recurrent thrombosis (21.1%; 7.9% recurrence on anticoagulation and 13.2% recurrence off anticoagulation) (P=0.001) (FIG. 1C). For patients with a known date of most recent thrombosis, the mHam was positive in 68.4% (13 of 19) of patients when thrombosis occurred within 1 year prior to testing, compared with 31.8% (7 of 22) of patients with their most recent thrombotic event more than 1 year prior to testing (P=0.019). A positive mHam assay >1 year after thrombosis was associated with recurrent VTE [85.7% (6 of 7), P=0.01 and triple-positivity [71.4% (5 of 7), P=0.0.

The mHam was positive in 43.8% (14 of 32) patients with positive anti-β2GPI antibodies compared with 25.9% (7 of 27) of patients who had only aPL other than anti-β2GPI antibodies (P=0.154). Among patient with anti-β2GPI antibodies), The mHam assay was positive in 50% (11 of 22) with anti-β2GPI IgG, 20% (1 of 5) with anti-β2GPI IgM, and 40% (2 of 5) with anti-β2GPI IgG and IgM (P=0.300).

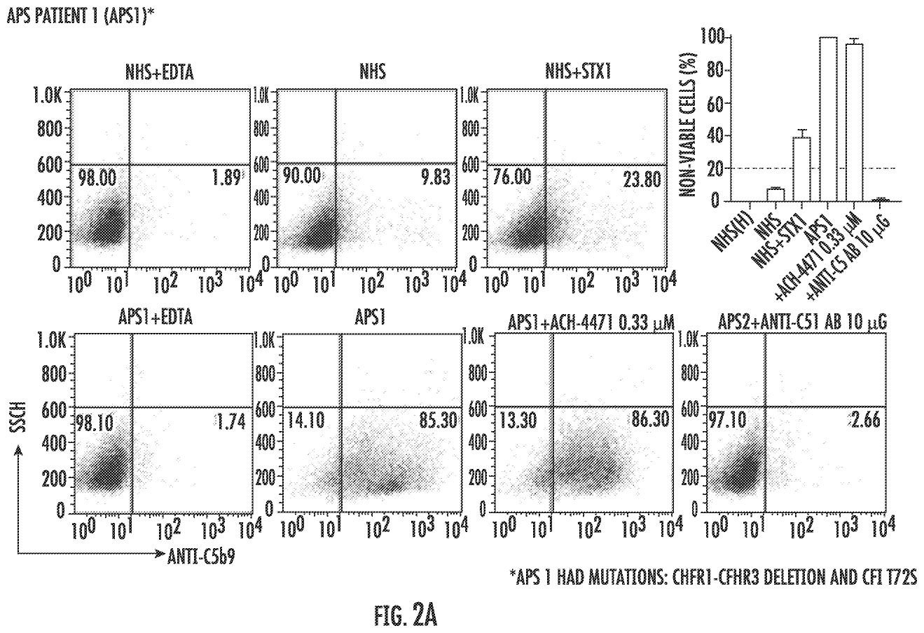

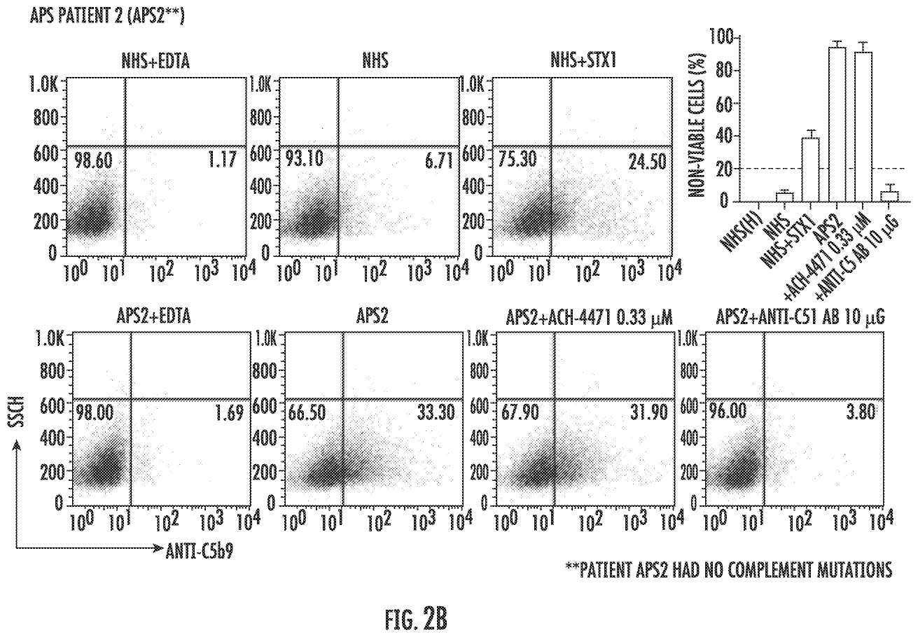

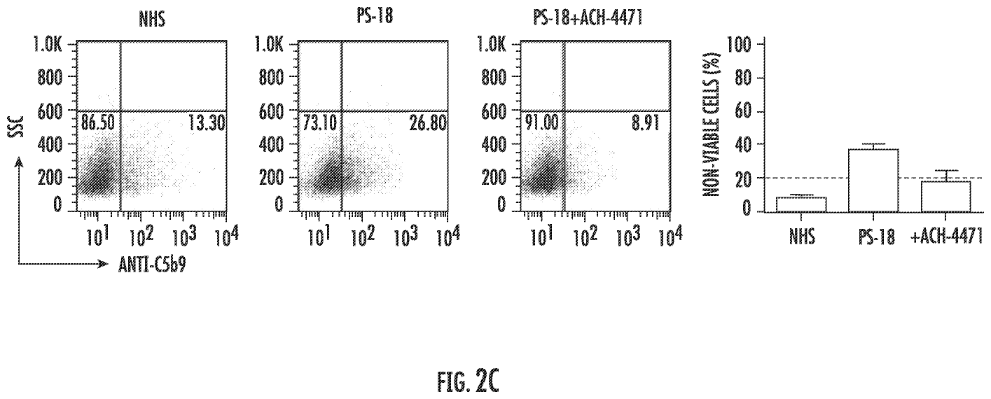

The present inventors evaluated deposition of terminal complement protein complexes (C5b-9) on PIGA null TF-1 cells following incubation with patient serum. APS patient sera induced C5b-9 deposition, which correlated with cell killing in the mHam assay (FIG. 2A). In all cases, C5b-9 deposition was inhibited by blocking the terminal pathway of complement with an anti-β5 antibody. The factor D inhibitor (ACH4471) specifically blocks the alternative pathway.⁴⁶ The present inventors previously demonstrated ACH4471 is highly effective in blocking complement dependent killing in the mHam with aHUS serum (FIG. 2B).⁴⁶ However, ACH4471 did not prevent cell killing in the mHam or prevent C5b-9 deposition induced by APS sera to any appreciable extent, suggesting that complement activation in APS sera primarily occurs through the classical pathway. In agreement, C5b-9 staining was not increased on the PIGA null TF1 cell line when incubated with sera in alternative pathway only buffer (GVB⁰-Mg-EGTA; data not shown) compared with a buffer that is not pathway-selective (GVB⁺⁺). Patients with a positive mHam demonstrated significantly higher mean C5b-9 deposition compared with negative controls (29.2% vs. 8.6%, P=0.006) while patients with a negative mHam did not show significantly increased mean C5b-9 deposition (12.6% versus 6.5%, P=0.331)

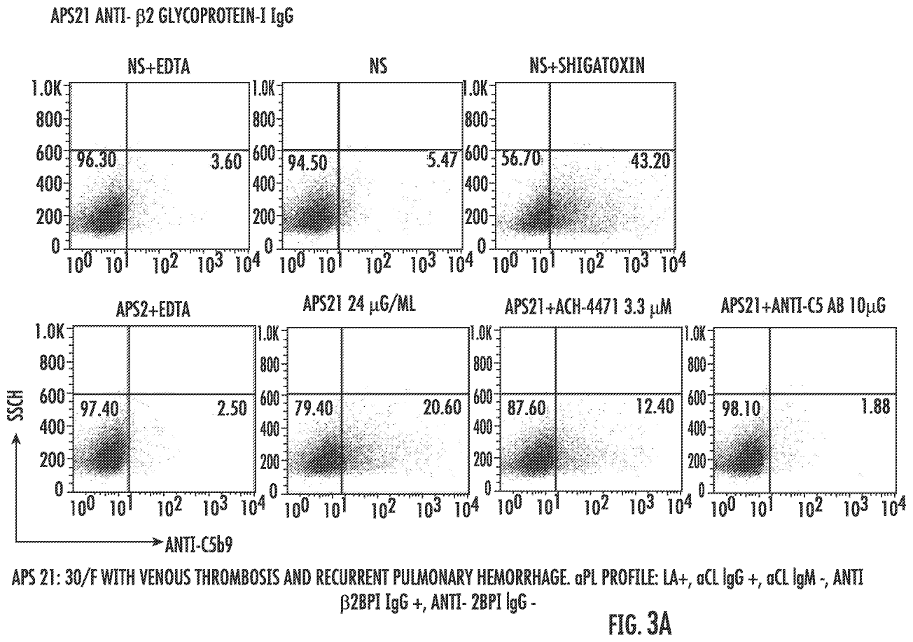

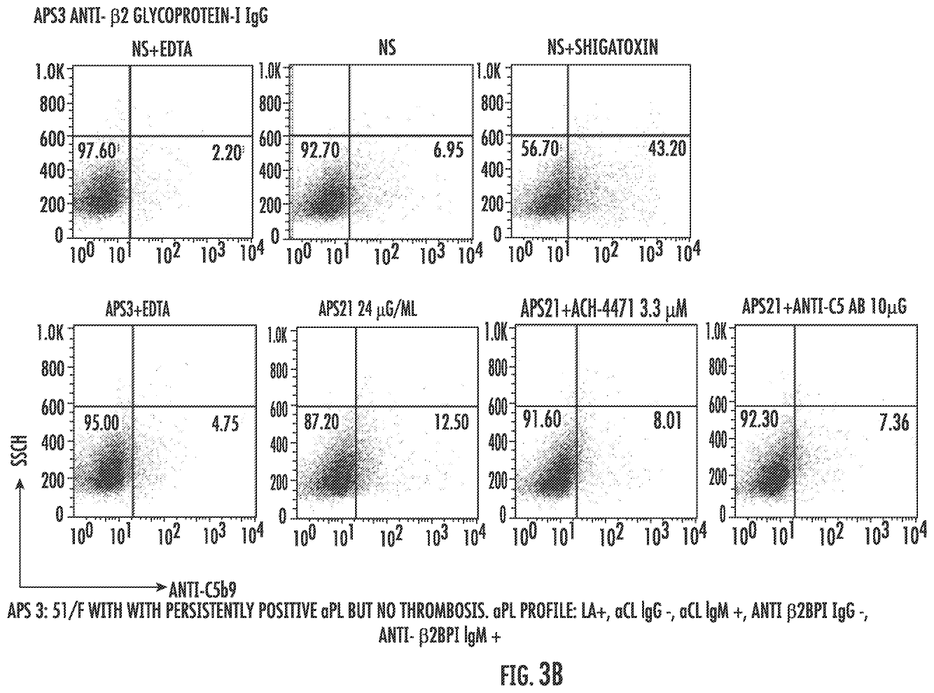

Patient-derived aPL activated complement in vitro. To further investigate whether aPL directly activate complement, the present inventors evaluated C5b-9 deposition on PIGA null TF1 cells incubated with serum to which affinity-purified, patient-specific anti-β₂GPI antibodies were added [one from a patient with thrombotic APS and triple-positive aPL profile including IgG anti-β₂GPI (APS21) and the other from a patient with a positive aPL profile including IgM anti-β₂GPI (and no IgG anti-β₂GPI) but no history of thrombosis (APS3)]. Anti-β₂GPI antibodies from the patient with thrombotic APS induced C5b-9 deposition, which was inhibited by adding anti-β5 monoclonal Ab and minimally inhibited by a factor D inhibitor (FIG. 3A). Anti-β₂GPI antibodies from the second patient (with positive LA, IgM anti-β₂GPI and IgM aCL and no thrombotic events) led to a very small increase in C5b-9 deposition (FIG. 3B) suggesting that complement activation is a characteristic of aPL that are associated with thrombosis.

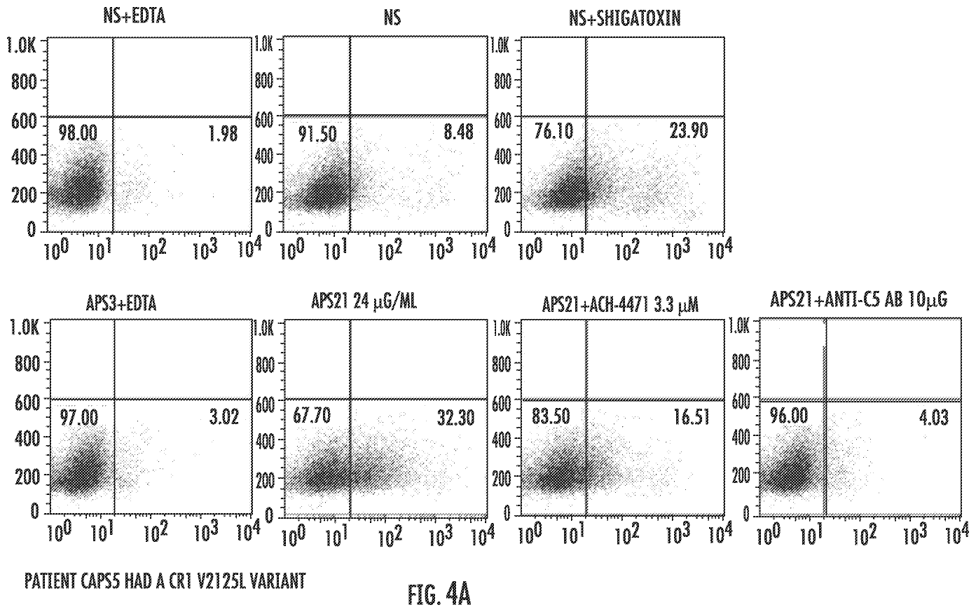

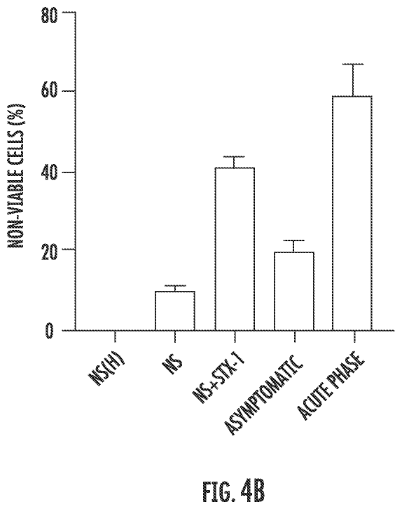

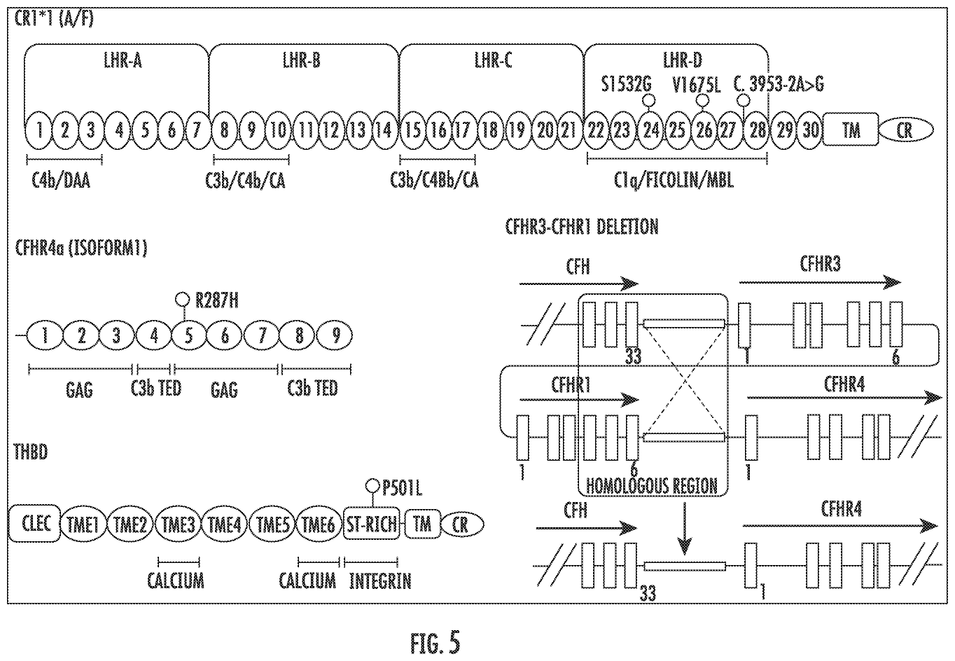

Catastrophic APS is associated with complement activation and rare germline variants in complement genes. The present inventors studied 10 patients with CAPS (Table 2). Acute phase sera were available for 7 patients and a positive mHam assay and increased C5b-9 deposition were detected in 85.7% (6 of 7). The only patient with a negative mHam assay during acute CAPS had a sample obtained after five plasma exchanges had been completed. Similar to APS, CAPS patients with positive mHam also demonstrated increased C5b-9 deposition on flow cytometry, which was blocked by eculizumab. C5b-9 deposition and mHam cell killing was partially blocked by ACH-4471 and completely blocked by anti-β5 monoclonal antibody (representative example in FIG. 4A).

Serial samples were available for two patients with CAPS. The first patient was a 40 year old male with a history of renal failure due to APS/CAPS who developed acute renal failure, cardiac injury, and thrombocytopenia within 2 weeks of a renal transplant, which was successfully treated with eculizumab. The mHam assay was positive during this acute episode but turned negative at 12 and 18 months after the acute episode. The second patient was a 36 year old female with a triple-positive APS with recurrent venous thromboembolic events despite anticoagulation, a history of hemolysis, elevated liver enzymes and low platelets (HELLP) syndrome during pregnancy, as well as two prior episodes of CAPS in the post-partum setting. She was evaluated during her fourth pregnancy at 17 weeks of gestation (at which time she was asymptomatic) and the mHam assay was negative. However, a repeat mHam at 23 weeks of gestation was positive (FIG. 4B). Two days after the second sample was drawn, she developed renal failure, thrombocytopenia and elevated transaminases and was diagnosed with recurrent CAPS/HELLP. Clinical deterioration continued even after emergent cesarean section. She was subsequently treated with plasma exchange and high dose corticosteroids in addition to anticoagulation and ultimately recovered after a prolonged, complicated hospital course. The patient was not treated with eculizumab. Her pattern of severe thrombotic illness in the latter half of pregnancy may be explained by the fact that complement activity normally rises late in pregnancy,⁴⁹ and this could serve as the ‘trigger’ in this case.

Germline variants in genes critical for alternative pathway of complement function and regulation contribute to complement-mediated diseases such as aHUS and HELLP.^{42,5}° The present inventors hypothesized that patients with APS and CAPS may harbor germline variants in complement genes as well, and performed target sequencing of the exonic regions of 15 genes with a role in the alternative pathway of complement or previously implicated in the etiology of aHUS. Genomic DNA samples were available from 10 patients with CAPS, 55 patients with APS and 21 patients with SLE (without aPL). To validate our custom sequencing panel, 33 patients with aHUS and 43 individuals without a diagnosis of a complement-mediated disease were sequenced as positive and negative controls, respectively. Rare germline variants were present in 60.0% (6 of 10) patients with CAPS and 51.5% (17 of 33) of aHUS, compared to 21.8% (12 of 55) patients with thrombotic APS, 28.6% (6 of 21) of SLE, and 23.3% (10 of 43) of unaffected individuals (Table 1). The frequency of germline variants in CAPS was significantly higher than in APS (P=0.013), SLE (without aPL) (P=0.093), and controls (P=0.022) and similar to that seen in aHUS (P=0.222). Rare variants in CAPS included: (i) homozygous CFHR1-CFHR3 deletion (2 patients), (ii) THBD P501L, (iii) CR1 S1532G and homozygous CFHR1-CFHR3 deletion, (iv) CFHR4 R287H, and a CR1 splice variant (v) CR1 V2125L (FIG. 5).

TABLE 1
Summary of rare variants in complement genes

| | Number of | Patients with rare | Mutation |
|---|---|---|---|
| | patients sequenced | variants* | rate |
| CAPS | 10 | 6 | 60.0% |
| APS | 55 | 12 | 21.8% |
| SLE | 21 | 6 | 28.6% |
| aHUS | 33 | 17 | 51.5% |
| Controls | 43 | 10 | 23.3% |

Genes included on panel: CFH, CFI, CFB, CFD, CFP, CFHR1, CFHR2, CFHR3, CFHR4, CFHR5, CR1, C3,THBD, CD46, DGKE.
*Rare variant was defined as minor allele frequency <0.005

5 Table 2.
Clinical characteristics, outcomes & complement studies in patients with CAPS

| | | | Definite/ | Prior | | | Repeat aPL | | |
|---|---|---|---|---|---|---|---|---|---|
| | Age/ | | Probable | APS | | | profile* (at least 12 | mHam | |
| S. # | Sex | Presentation | CAPS | diagnosis | Trigger | aPL profile* | weeks apart) | positive | Mutations^{†} |
| 1 | 45/M | Ischemic toe | Probable | + | ITP treatment | DRVVT+, hexagonal | DRVVT+, | n/a | None |
| | | after initiation | | | (eltrombopag) | PL+, PNP+, aβ2GPI | hexagonal PL+, | | |
| | | of ITP | | | | IgG 52, aβ2GPI IgM 34, | PNP+, aβ2GPI IgG | | |
| | | therapy, DVT, | | | | aCL IgG 126, aCL IgM | >150, aβ2GPI IgM | | |
| | | pulmonary | | | | 24 | 52, aCL IgG >150, | | |
| | | embolism, | | | | | aCL IgM 38 | | |
| | | myocardial | | | | | | | |
| | | infarction, | | | | | | | |
| | | renal | | | | | | | |
| | | insufficiency | | | | | | | |
| 2 | 37/F | Subdural | Probable | + | Myocarditis, | DRVVT+, hexagonal | DRVVT+, | n/a | None |
| | | hematoma, | | | possibly viral | PL+. PNP+, aβ2GPI | hexagonal PL+, | | |
| | | cardiomyopathy, | | | | >150, aβ2GPI IgM 146, | PNP+, aβ2GPI | | |
| | | acute | | | | aCL IgG 127, | >107, aβ2GPI IgM | | |
| | | kidney injury, | | | | aCL IgM 69 | >150, aCL IgG 121 | | |
| | | thrombocytopenia | | | | | aCL IgM 120 | | |
| 3 | 40/M | Renal failure | Definite | + | Acute | DRVVT+, aCL IgG | DRVVT+, aCL IgG | + | THBD |
| | | (biopsy | | | appendicitis | <10, aCL IgM <10, | <10, aCL IgM <10, | | |
| | | showed | | | | aβ2GPI IgG <10, | aβ2GPI IgG <10, | | |
| | | cortical | | | | aβ2GPI IgM <10 | aβ2GPI IgM <10 | | |
| | | thrombosis | | | | | | | |
| | | and TMA), | | | | | | | |
| | | thrombocytopenia, | | | | | | | |
| | | pulmonary | | | | | | | |
| | | embolism | | | | | | | |
| 4 | 67/F | Pulmonary | Probable | − | Rheumatoid | DRVVT+, aCL IgG | Repeat was not | + | CFHR1- |
| | | emboli, | | | arthritis flare | <10, aCL IgM <10, | obtained | | CFHR3 del |
| | | arterial | | | | aβ2GPI IgG <10, | | | (homozygous) |
| | | thrombosis | | | | aβ2GPI IgM <10 | | | |
| | | requiring leg | | | | | | | |
| | | amputation | | | | | | | |
| | | (pathology | | | | | | | |
| | | showed | | | | | | | |
| | | widespread | | | | | | | |
| | | small vessel | | | | | | | |
| | | thrombosis), | | | | | | | |
| | | digital | | | | | | | |
| | | gangrene, | | | | | | | |
| | | renal failure | | | | | | | |
| 5 | 34/F | Renal failure, | Probable | + | Pregnancy | DRVVT+, aCL IgG 131 | DRVVT+, | + | CR1 |
| | | thrombocytop | | | | | aCL IgG 64 | | |
| | | enia and | | | | | | | |
| | | elevated liver | | | | | | | |
| | | enzymes | | | | | | | |
| 6 | 63/M | Thrombocyto | Definite | + | Pyelonephritis | DRVVT+, aCL IgG | DRVVT+, aCL IgG | + | CFHR4, |
| | | penia, renal | | | | <10, aCL IgM 28, | <10, aCL IgM 28, | | CR1 |
| | | failure, | | | | aβ2GPI IgG31, | aβ2GPI IgG 41, | | |
| | | cardiomyopathy | | | | aβ2GPI IgM <10 | aβ2GPI IgM <10 | | |
| | | with | | | | | | | |
| | | reduced | | | | | | | |
| | | ejection | | | | | | | |
| | | fraction, | | | | | | | |
| | | adrenal | | | | | | | |
| | | hemorrhage, | | | | | | | |
| | | skin ulcers. | | | | | | | |
| | | Renal biopsy | | | | | | | |
| | | showed TMA. | | | | | | | |
| 7 | 49/F | Arterial | Probable | + | None | DRVVT+, aCL IgG 62, | Note repeated after | − | CR1 |
| | | thrombosis of | | | | aCL IgM 66, aβ2GPI | CAPS episode since | | |
| | | lower | | | | IgG <10, | patient died. | | |
| | | extremities, | | | | aβ2GPI IgM 40 | Previous testing (>1 | | |
| | | renal failure, | | | | | year prior) showed: | | |
| | | cardiac | | | | | DRVVT+, aCL IgG | | |
| | | ischemia, | | | | | 21, aCL IgM 25, | | |
| | | gangrene of | | | | | aβ2GPI IgG 31, | | |
| | | fingertips, | | | | | aβ2GPI IgM 15 | | |
| | | thrombocytopenia, | | | | | | | |
| | | skin | | | | | | | |
| | | necrosis | | | | | | | |
| 8 | 50/M | Hepatic, | Probable | + | Acute | DRVVT+, aCL IgG | DRVVT+, aCL IgG | + | None |
| | | spleen and | | | cholecystitis | >100, aCL IgM <10, | >100, aCL IgM | | |
| | | retinal | | | and | aβ2GPI IgG >100, | <10, aβ2GPI IgG | | |
| | | infarction, | | | cholecystectomy | aβ2GPI IgM 14 | >100, | | |
| | | renal failure | | | | | aβ2GPI IgM 14 | | |
| 9 | 46/F | Diffuse | Probable | − | None | DRVVT+, aCL IgG | aCL IgG 52.5 (on | + | CFHR1- |
| | | hepatic | | | | 143, aCL IgM 73, | plasmapheresis), | | CFHR3 del |
| | | infarction and | | | | aβ2GPI IgG 61 | DRVVT not | | (homozygous) |
| | | evidence of | | | | | repeated due to | | |
| | | microvascular | | | | | ongoing | | |
| | | thrombosis in | | | | | anticoagulation | | |
| | | the lungs, | | | | | | | |
| | | spleen and | | | | | | | |
| | | possible | | | | | | | |
| | | kidney. | | | | | | | |
| | | Relapsed | | | | | | | |
| | | about 6 | | | | | | | |
| | | months after | | | | | | | |
| | | initial | | | | | | | |
| | | presentation | | | | | | | |
| | | with recurrent | | | | | | | |
| | | hepatic | | | | | | | |
| | | infarcts. | | | | | | | |
| 10 | 51/M | Multiple | Definite | + | Mitral valve | DRVVT+, aCL IgG | DRVVT+, aCL IgG | n/a | None |
| | | thrombi on | | | replacement | <10, aCL IgM <10, | <10, aCL IgM<10, | | |
| | | mitral valve, | | | | aβ2GPI IgG <10, | aβ2GPI IgG<10, | | |
| | | renal failure, | | | | aβ2GPI IgM <10 | aβ2GPI IgM<10 | | |
| | | infarcts and | | | | | | | |
| | | ischemic | | | | | | | |
| | | injury of liver | | | | | | | |
| | | and spleen, | | | | | | | |
| | | multiple small | | | | | | | |
| | | vessel strokes | | | | | | | |
| 1 | 45/M | Ischemic toe | Probable | + | ITP treatment | DRVVT+, hexagonal | DRVVT+, | n/a | None |
| | | after initiation | | | (eltrombopag) | PL+, PNP+, aβ2GPI | hexagonal PL+, | | |
| | | of ITP | | | | IgG 52, aβ2GPI IgM 34, | PNP+, aβ2GPI IgG | | |
| | | therapy, DVT, | | | | aCL IgG 126, aCL IgM | >150, aβ2GPI IgM | | |
| | | pulmonary | | | | 24 | 52, aCL IgG >150, | | |
| | | embolism, | | | | | aCL IgM 38 | | |
| | | myocardial | | | | | | | |
| | | infarction, | | | | | | | |
| | | renal | | | | | | | |
| | | insufficiency | | | | | | | |
| 2 | 37/F | Subdural | Probable | + | Myocarditis, | DRVVT+, hexagonal | DRVVT+, | n/a | None |
| | | hematoma, | | | possibly viral | PL+. PNP+, aβ2GPI | hexagonal PL+, | | |
| | | cardiomyopathy, | | | | >150, aβ2GPI IgM 146, | PNP+, aβ2GPI | | |
| | | acute | | | | aCL IgG 127, aCL IgM | >107, aβ2GPI IgM | | |
| | | kidney injury, | | | | 69 | >150, aCL IgG 121 | | |
| | | thrombocytopenia | | | | | aCL IgM 120 | | |
| 3 | 40/M | Renal failure | Definite | + | Acute | DRVVT+, aCL IgG | DRVVT+, aCL IgG | + | THBD |
| | | (biopsy | | | appendicitis | <10, aCL IgM <10, | <10, aCL IgM <10, | | |
| | | showed | | | | aβ2GPI IgG <10, | aβ2GPI IgG <10, | | |
| | | cortical | | | | aβ2GPI IgM <10 | aβ2GPI IgM <10 | | |
| | | thrombosis | | | | | | | |
| | | and TMA), | | | | | | | |
| | | thrombocytopenia, | | | | | | | |
| | | pulmonary | | | | | | | |
| | | embolism | | | | | | | |
| 4 | 67/F | Pulmonary | Probable | − | Rheumatoid | DRVVT+, aCL IgG | Repeat was not | + | CFHR1- |
| | | emboli, | | | arthritis flare | <10, aCL IgM <10, | obtained | | CFHR3 del |
| | | arterial | | | | aβ2GPI IgG <10, | | | (homozygous) |
| | | thrombosis | | | | aβ2GPI IgM <10 | | | |
| | | requiring leg | | | | | | | |
| | | amputation | | | | | | | |
| | | (pathology | | | | | | | |
| | | showed | | | | | | | |
| | | widespread | | | | | | | |
| | | small vessel | | | | | | | |
| | | thrombosis), | | | | | | | |
| | | digital | | | | | | | |
| | | gangrene, | | | | | | | |
| | | renal failure | | | | | | | |
| 5 | 34/F | Renal failure, | Probable | + | Pregnancy | DRVVT+, aCL IgG 131 | DRVVT+, aCL IgG | + | CR1 |
| | | thrombocytopenia | | | | | 64 | | |
| | | and | | | | | | | |
| | | elevated liver | | | | | | | |
| | | enzymes | | | | | | | |
| 6 | 63/M | Thrombocytopenia, | Definite | + | Pyelonephritis | DRVVT+, aCL IgG | DRVVT+, aCL IgG | + | CFHR4, |
| | | renal | | | | <10, aCL IgM 28, | <10, aCL IgM 28, | | CR1 |
| | | failure, | | | | aβ2GPI IgG31, | aβ2GPI IgG41, | | |
| | | cardiomyopathy | | | | aβ2GPI IgM <10 | aβ2GPI IgM <10 | | |
| | | with | | | | | | | |
| | | reduced | | | | | | | |
| | | ejection | | | | | | | |
| | | fraction, | | | | | | | |
| | | adrenal | | | | | | | |
| | | hemorrhage, | | | | | | | |
| | | skin ulcers. | | | | | | | |
| | | Renal biopsy | | | | | | | |
| | | showed TMA. | | | | | | | |
| 7 | 49/F | Arterial | Probable | + | None | DRVVT+, aCL IgG 62, | Note repeated after | − | CR1 |
| | | thrombosis of | | | | aCL IgM 66, aβ2GPI | CAPS episode since | | |
| | | lower | | | | IgG <10, aβ2GPI IgM | patient died. | | |
| | | extremities, | | | | 40 | Previous testing (>1 | | |
| | | renal failure, | | | | | year prior) showed: | | |
| | | cardiac | | | | | DRVVT+, aCL IgG | | |
| | | ischemia, | | | | | 21, aCL IgM 25, | | |
| | | gangrene of | | | | | aβ2GPI IgG 31, | | |
| | | fingertips, | | | | | aβ2GPI IgM 15 | | |
| | | thrombocytopenia, | | | | | | | |
| | | skin | | | | | | | |
| | | necrosis | | | | | | | |
| 8 | 50/M | Hepatic, | Probable | + | Acute | DRVVT+, aCL IgG | DRVVT+, aCL IgG | + | None |
| | | spleen and | | | cholecystitis | >100, aCL IgM <10, | >100, aCL IgM | | |
| | | retinal | | | and | aβ2GPI IgG >100, | <10, aβ2GPI IgG | | |
| | | infarction, | | | cholecystectomy | aβ2GPI IgM 14 | >100, aβ2GPI IgM | | |
| | | renal failure | | | | | 14 | | |
| 9 | 46/F | Diffuse | Probable | − | None | DRVVT+, aCL IgG | aCL IgG 52.5 (on | + | CFHR1- |
| | | hepatic | | | | 143, aCL IgM 73, | plasmapheresis), | | CFHR3 del |
| | | infarction and | | | | aβ2GPI IgG 61 | DRVVT not | | (homozygous) |
| | | evidence of | | | | | repeated due to | | |
| | | microvascular | | | | | on going | | |
| | | thrombosis in | | | | | anticoagulation | | |
| | | the lungs, | | | | | | | |
| | | spleen and | | | | | | | |
| | | possible | | | | | | | |
| | | kidney. | | | | | | | |
| | | Relapsed | | | | | | | |
| | | about 6 | | | | | | | |
| | | months after | | | | | | | |
| | | initial | | | | | | | |
| | | presentation | | | | | | | |
| | | with recurrent | | | | | | | |
| | | hepatic | | | | | | | |
| | | infarcts. | | | | | | | |
| 10 | 51/M | Multiple | Definite | + | Mitral valve | DRVVT+, aCL IgG | DRVVT+, aCL IgG | n/a | None |
| | | thrombi on | | | replacement | <10, aCL IgM <10, | <10, aCL IgM <10, | | |
| | | mitral valve, | | | | aβ2GPI IgG <10, | aβ2GPI IgG <10, | | |
| | | renal failure, | | | | aβ2GPI IgM <10 | aβ2GPI IgM <10 | | |
| | | infarcts and | | | | | | | |
| | | ischemic | | | | | | | |
| | | injury of liver | | | | | | | |
| | | and spleen, | | | | | | | |
| | | multiple small | | | | | | | |
| | | vessel strokes | | | | | | | |

### Discussion

APS serum demonstrates complement activation shown by a functional assay (mHam) and increased C5b-9 deposition on the cell surface, which is recapitulated on adding patient-derived anti-132GPI antibodies to normal sera. CAPS patients have a high rate of rare germline variants in complement regulatory genes, which may serve as a ‘second-hit’ (in addition to aPL) leading to uncontrolled complement activation and a more severe clinical phenotype.

Though the aPL profile helps risk stratify patients with APS,²¹ the absence of a reliable biomarker to distinguish clinically-relevant from clinically-irrelevant aPL (and to predict which patients will have first or recurrent thrombosis) is a major gap in APS clinical care. Previous studies have demonstrated hypocomplementemia³⁶ and increased levels of complement activation byproducts including fragment Bb, C3a, and C5b-9 in patients with APS³³⁻³⁵ but did not consistently show an association with thrombosis.^{35,36} Serum levels of complement byproducts measure complement activation indirectly, and it is difficult to establish clinically relevant thresholds on these assays. A functional assay was used to show that complement activation is associated with patients with aPL who had thrombosis, and patients with a more severe APS phenotype characterized by recurrent thrombosis are more likely to have evidence of persistent complement activation. Approximately 35% of patients with thrombotic APS had a positive mHam assay compared with 6.8% of controls with SLE. The mHam assay was positive in 68.4% of patients enrolled within a year of their thrombotic events compared with 31.8% in patients enrolled beyond one year. Most patients with positive mHam assays beyond a year after their thrombotic event had had recurrent thrombosis and a triple-positive aPL profile. The present findings suggest that complement activation as measured in our assay may be a marker of more clinically important APS. The mHam was positive in a higher proportion of patients with anti-β2GPI aPL compared with patients with other aPL (43.8% vs. 25.9%, P=0.154). Additionally, the mHam was more likely to be positive in patients with IgG anti-β2GPI (50%) or both IgG and IgM anti-β2GPI (40%) than those with IgM anti-β2GPI alone (20%) (P=0.467). However, these differences were not statistically significant, likely because of the small number of patients in these subgroups. This trend is consistent with the stronger association of IgG aPL with thrombotic events.⁵¹

Anti-β2GPI IgG from patients with thrombotic APS lead to membrane attack complex (C5b-9) deposition on the cell surface, which echoes murine models of APS in which aPL induced deposition of complement proteins on the vascular endothelium and thrombosis was attenuated in C6 deficient (C6 −/−) rats or animals treated with a CS inhibitor.¹⁶ Meroni et al. also showed that C5b-9 co-localized with β2GPI and IgG in the arterial wall of a patient with APS and recurrent arterial thrombosis who responded to treatment with eculizumab.²⁸ In contrast, anti-β2GPI IgM from a patient without thrombosis did not activate complement highlighting that complement activation is a critical characteristic of pathogenic aPL. Agostinis et al. previously showed that a non-complement fixing version of an anti-β2GPI antibody lost the ability to induce thrombosis in a rat model.¹⁷ The link between complement activation and thrombosis is illustrated by experiments showing that aPL induced complement activation leads to tissue factor dependent procoagulant activity due to the effects of C5a on neutrophils,⁵² monocytes⁵³ and endothelial cells⁵⁴ as well endothelial tissue factor expression induced by C5b-9.⁵⁵

The mechanisms by which anti-β2GPI antibodies cause complement activation remain unclear; however, mechanisms involving autoantibodies against factor 14,⁵⁶⁻⁵⁸ a regulatory role of cell surface bound β2GPI,⁵⁹ and activation of the classical complement pathway through Clq have been proposed.⁶⁰ The present inventors found that complement activation (cell killing and C5b-9 deposition) in APS sera or induced by patient-derived aPL was blocked by a terminal complement inhibitor (anti-CS antibody) and less so by an alternative pathway selective inhibitor (ACH-4471) suggesting that aPL induces complement activation primarily through the classical complement pathway. In contrast, complement dependent cell killing and C5b-9 deposition induced by CAPS sera were partially inhibited by ACH-4471 (and more so by anti-CS antibody) suggesting activation of both the classical and alternative pathways of complement. This may be explained by the higher rate of mutations in genes regulating the alternative complement pathway in patients with CAPS.

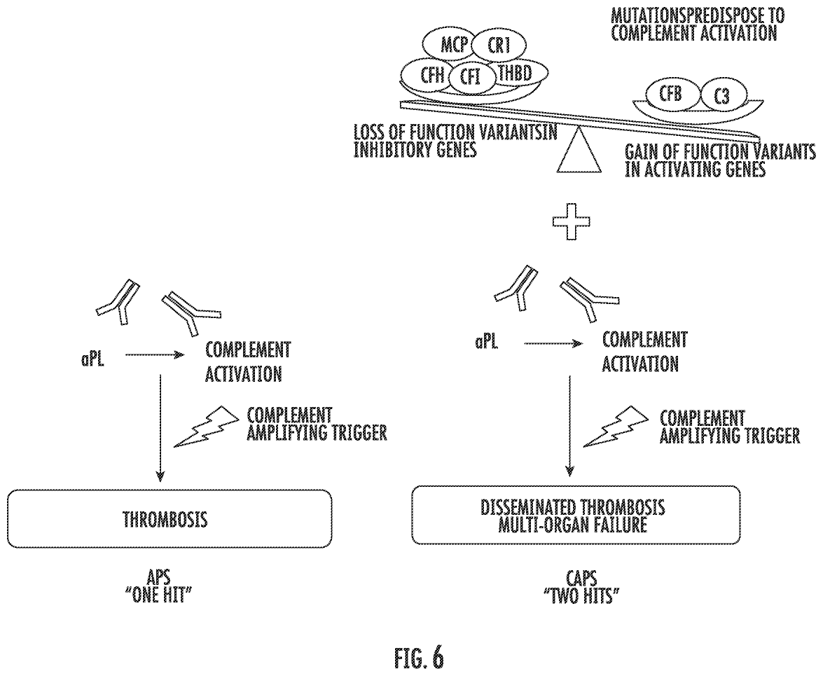

Rare germline variants in complement genes were detected in 60% of patients with CAPS. Acute CAPS was also characterized by complement activation highlighting the clinical and pathogenic similarities between CAPS and aHUS, the prototypical complement-mediated microangiopathy.⁶¹ The present inventors recently demonstrated complement activation and germline complement gene variants in patients with the HELLP syndrome,⁴² which is several-fold more common in patients with APS and exhibits marked clinical overlap with CAPS.⁶² The present inventors propose a pathogenic model in which aPL are the ‘first-hit’ that can induce complement activation and cause thrombosis, while patients who also have a pathogenic complement regulatory gene mutation (“second-hit”) are predisposed to uncontrolled complement activation, leading to CAPS in the setting of a complement amplifying trigger such as infection, surgery, pregnancy, or autoimmune disease (FIG. 6).⁶³ Several reports describe the successful use of eculizumab to treat CAPS,²⁹⁻³² and thrombotic APS refractory to anticoagulation;²⁸ however, mechanistic data and disease biomarkers have been lacking. The present findings provide a rationale for studying complement inhibition as a therapeutic strategy for CAPS and anticoagulation-refractory APS.

Limitations of the described study include that a highly selected group of patients who were referred to tertiary care centers that may not be fully representative of all APS patients was evaluated. Patients were not recruited consecutively and were not matched to the control group (SLE) for treatments or comorbidities. Time from event to sampling was variable and we did not have serial samples from enough patients to draw robust conclusions regarding the persistence of complement activation over time. Finally, the present inventors do not currently have functional data other than the mHam to confirm the pathogenic significance of these germline variants; however, their frequency is comparable to that found in patients with aHUS⁶¹ and significantly higher than control groups without TMA.

In summary, anti-β2GPI from patients with APS activate complement, and complement activation correlates with thrombotic events in APS. Moreover, the majority of patients with CAPS have underlying complement regulatory gene variants that predispose to increased complement activity and a fulminant course with widespread thrombosis and multi-organ failure.

### REFERENCES

1. Miyakis S, Lockshin M D, Atsumi T, et al. International consensus statement on an update of the classification criteria for definite antiphospholipid syndrome (APS). J Thromb Haemost. 2006; 4(2):295-306.
2. Galli M, Borrelli G, Jacobsen E M, et al. Clinical significance of different antiphospholipid antibodies in the WAPS (warfarin in the antiphospholipid syndrome) study. Blood. 2007; 110(4):1178-1183.
3. McNeil H P, Simpson R J, Chesterman C N, Krilis S A. Anti-phospholipid antibodies are directed against a complex antigen that includes a lipid-binding inhibitor of coagulation: beta 2-glycoprotein I (apolipoprotein H). Proc Natl Acad Sci USA. 1990; 87(11):4120-4124.
4. Arad A, Proulle V, Furie R A, Furie B C, Furie B. beta(2)-Glycoprotein-1 autoantibodies from patients with antiphospholipid syndrome are sufficient to potentiate arterial thrombus formation in a mouse model. Blood. 2011; 117(12):3453-3459.
5. Marciniak E, Romond E H. Impaired catalytic function of activated protein C: a new in vitro manifestation of lupus anticoagulant. Blood. 1989; 74(7):2426-2432.
6. Urbanus R T, de Laat B. Antiphospholipid antibodies and the protein C pathway. Lupus. 2010; 19(4):394-399.
7. Liestol S, Sandset P M, Jacobsen E M, Mowinckel M C, Wisloff F. Decreased anticoagulant response to tissue factor pathway inhibitor type 1 in plasmas from patients with lupus anticoagulants. Br J Haematol. 2007; 136(1):131-137.
8. Bu C, Gao L, Xie W, et al. beta2-glycoprotein i is a cofactor for tissue plasminogen activator-mediated plasminogen activation. Arthritis Rheum. 2009; 60(2):559-568.
9. Zhang J, McCrae K R. Annexin A2 mediates endothelial cell activation by antiphospholipid/anti-beta2 glycoprotein I antibodies. Blood. 2005; 105(5):1964-1969.
10. Shi T, Giannakopoulos B, Yan X, et al. Anti-beta2-glycoprotein I antibodies in complex with beta2-glycoprotein I can activate platelets in a dysregulated manner via glycoprotein Ib-IX-V. Arthritis Rheum. 2006; 54(8):2558-2567.
11. Sorice M, Longo A, Capozzi A, et al. Anti-beta2-glycoprotein I antibodies induce monocyte release of tumor necrosis factor alpha and tissue factor by signal transduction pathways involving lipid rafts. Arthritis Rheum. 2007; 56(8):2687-2697.
12. Chaturvedi S, Alluri R, McCrae K R. Extracellular Vesicles in the Antiphospholipid Syndrome. Semin Thromb Hemost. 2018; 44(5):493-504.
13. Rand J H, Wu X X, Guller S, et al. Reduction of annexin-V (placental anticoagulant protein-I) on placental villi of women with antiphospholipid antibodies and recurrent spontaneous abortion. Am J Obstet Gynecol. 1994; 171(6):1566-1572.
14. Pierangeli S S, Girardi G, Vega-Ostertag M, Liu X, Espinola R G, Salmon J. Requirement of activation of complement C3 and C5 for antiphospholipid antibody-mediated thrombophilia. Arthritis Rheum. 2005; 52(7):2120-2124.
15. Carrera-Marin A, Romay-Penabad Z, Papalardo E, et al. C6 knock-out mice are protected from thrombophilia mediated by antiphospholipid antibodies. Lupus. 2012; 21(14):1497-1505.
16, Fischetti F, Durigutto P, Pellis V, et al. Thrombus formation induced by antibodies to beta2-glycoprotein I is complement dependent and requires a priming factor. Blood. 2005; 106(7):2340-2346.
17. Agostinis C, Durigutto P, Sblattero D, et al. A non-complement-fixing antibody to beta2 glycoprotein I as a novel therapy for antiphospholipid syndrome. Blood. 2014; 123(22):3478-3487.
18. Girardi G, Berman J, Redecha P, et al. Complement C5a receptors and neutrophils mediate fetal injury in the antiphospholipid syndrome. J Clin Invest. 2003; 112(11):1644-1654.
19. Girardi G, Redecha P, Salmon J E. Heparin prevents antiphospholipid antibody-induced fetal loss by inhibiting complement activation. Nat Med. 2004; 10(11):1222-1226.
20. Redecha P, Tilley R, Tencati M, et al. Tissue factor: a link between C5a and neutrophil activation in antiphospholipid antibody induced fetal injury. Blood. 2007; 110(7):2423-2431.
21. de Groot P G, Lutters B, Derksen R H, Lisman T, Meijers J C, Rosendaal F R. Lupus anticoagulants and the risk of a first episode of deep venous thrombosis. J Thromb Haemost. 2005; 3(9):1993-1997.
22. Galli M, Luciani D, Bertolini G, Barbui T. Lupus anticoagulants are stronger risk factors for thrombosis than anticardiolipin antibodies in the antiphospholipid syndrome: a systematic review of the literature. Blood. 2003; 101(5):1827-1832.
23. Pengo V, Ruffatti A, Legnani C, et al. Incidence of a first thromboembolic event in asymptomatic carriers of high-risk antiphospholipid antibody profile: a multicenter prospective study. Blood. 2011; 118(17):4714-4718.
24. Lee E Y, Lee C K, Lee T H, et al. Does the anti-beta2-glycoprotein I antibody provide additional information in patients with thrombosis? Thromb Res. 2003; 111(1-2):29-32.
25. Chaturvedi S, McCrae K R. Clinical Risk Assessment in the Antiphospholipid Syndrome: Current Landscape and Emerging Biomarkers. Curr Rheumatol Rep. 2017; 19(7):43.
26. Cervera R, Serrano R, Pons-Estel G J, et al. Morbidity and mortality in the antiphospholipid syndrome during a 10-year period: a multicentre prospective study of 1000 patients. Ann Rheum Dis. 2015; 74(6):1011-1018.
27, Cervera R, Espinosa G. Update on the catastrophic antiphospholipid syndrome and the “CAPS Registry”. Semin Thromb Hemost. 2012; 38(4):333-338.
28. Meroni P L, Macor P, Durigutto P, et al. Complement activation in antiphospholipid syndrome and its inhibition to prevent rethrombosis after arterial surgery. Blood. 2016; 127(3):365-367.
29. Shapira I, Andrade D, Allen S L, Salmon J E. Brief report: induction of sustained remission in recurrent catastrophic antiphospholipid syndrome via inhibition of terminal complement with eculizumab. Arthritis Rheum. 2012; 64(8):2719-2723.
30. Wig S, Chan M, Thachil J, Bruce I, Barnes T. A case of relapsing and refractory catastrophic anti-phospholipid syndrome successfully managed with eculizumab; a complement 5 inhibitor. Rheumatology (Oxford). 2016; 55(2):382-384.
31. Zikos T A, Sokolove J, Ahuja N, Berube C. Eculizumab Induces Sustained Remission in a Patient With Refractory Primary Catastrophic Antiphospholipid Syndrome. J Clin Rheumatol. 2015; 21(6):311-313.
32. Strakhan M, Hurtado-Sbordoni M, Galeas N, Bakirhan K, Alexis K, Elrafei T. 36-year-old female with catastrophic antiphospholipid syndrome treated with eculizumab: a case report and review of literature. Case Rep Hematol. 2014; 2014:704371.
33. Davis W D, Brey R L. Antiphospholipid antibodies and complement activation in patients with cerebral ischemia. Clin Exp Rheumatol. 1992; 10(5):455-460.
34. Breen K A, Seed P, Parmar K, Moore G W, Stuart-Smith S E, Hunt B J. Complement activation in patients with isolated antiphospholipid antibodies or primary antiphospholipid syndrome. Thromb Haemost. 2012; 107(3):423-429.
35. Devreese K M, Hoylaerts M F. Is there an association between complement activation and antiphospholipid antibody-related thrombosis? Thromb Haemost. 2010; 104(6):1279-1281.
36. Oku K, Atsumi T, Bohgaki M, et al. Complement activation in patients with primary antiphospholipid syndrome. Ann Rheum Ms. 2009; 68(6):1030-1035.
37. Fangtham M, Petri M. 2013 update: Hopkins lupus cohort. Curr Rheumatol Rep. 2013; 15(9):360.
38. Asherson R A, Cervera R, de Groot P G, et al. Catastrophic antiphospholipid syndrome: international consensus statement on classification criteria and treatment guidelines. Lupus. 2003; 12(7):530-534.
39. Pineton de Chambrun M, Larcher R, Pene F, et al. CAPS criteria fail to identify most severely-ill thrombotic antiphospholipid syndrome patients requiring intensive care unit admission. J Autoimmun. 2019; 103:102292.
40. Legault K, Schunemann H, Hillis C, et al. McMaster RARE-Bestpractices clinical practice guideline on diagnosis and management of the catastrophic antiphospholipid syndrome. J Thromb Haemost. 2018.
41. Petri M, Orbai A M, Alarcon G S, et al. Derivation and validation of the Systemic Lupus International Collaborating Clinics classification criteria for systemic lupus erythematosus. Arthritis Rheum. 2012; 64(8):2677-2686.
42. Vaught A J, Braunstein E M, Jasem J, et al. Germline mutations in the alternative pathway of complement predispose to HELLP syndrome. JCI Insight. 2018; 3(6).
43. Ma K, Simantov R, Zhang J C, Silverstein R, Hajjar K A, McCrae K R. High affinity binding of beta 2-glycoprotein I to human endothelial cells is mediated by annexin II. J Biol Chem. 2000; 275(20):15541-15548.
44. Gavriilaki E, Yuan X, Ye Z, et al. Modified Ham test for atypical hemolytic uremic syndrome. Blood. 2015; 125(23):3637-3646.
45. Brady T M, Pruette C, Loeffler L F, et al. Typical Hus: Evidence of Acute Phase Complement Activation from a Daycare Outbreak. J Clin Exp Nephrol. 2016; 1(2).
46. Yuan X, Gavriilaki E, Thanassi J A, et al. Small-molecule factor D inhibitors selectively block the alternative pathway of complement in paroxysmal nocturnal hemoglobinuria and atypical hemolytic uremic syndrome. Haematologica. 2017; 102(3):466-475.
47. Zipfel P F, Edey M, Heinen S, et al. Deletion of complement factor H-related genes CFHR1 and CFHR3 is associated with atypical hemolytic uremic syndrome. PLoS Genet. 2007; 3(3):e41.
48. Vaught A J, Gavriilaki E, Hueppchen N, et al. Direct evidence of complement activation in HELLP syndrome: A link to atypical hemolytic uremic syndrome. Exp Hematol. 2016; 44(5):390-398.
49. Derzsy Z, Prohaszka Z, Rigo J, Jr., Fust G, Molvarec A. Activation of the complement system in normal pregnancy and preeclampsia. Mol Immunol. 2010; 47(7-8):1500-1506.
50. Kavanagh D, Goodship T. Genetics and complement in atypical HUS. Pediatr Nephrol. 2010; 25(12):2431-2442.
51. Kelchtermans H, Pelkmans L, de Laat B, Devreese K M. IgG/IgM antiphospholipid antibodies present in the classification criteria for the antiphospholipid syndrome: a critical review of their association with thrombosis. J Thromb Haemost. 2016; 14(8):1530-1548.
52. Ritis K, Doumas M, Mastellos D, et al. A novel C5a receptor-tissue factor cross-talk in neutrophils links innate immunity to coagulation pathways. J Immunol. 2006; 177(7):4794-4802.
53. Wolberg A S, Roubey R A. Mechanisms of autoantibody-induced monocyte tissue factor expression. Thromb Res. 2004; 114(5-6):391-396.
54. Ikeda K, Nagasawa K, Horiuchi T, Tsuru T, Nishizaka H, Niho Y. C5a induces tissue factor activity on endothelial cells. Thromb Haemost. 1997; 77(2):394-398.
55. Saadi S, Holzknecht R A, Patte C P, Stem D M, Platt J L. Complement-mediated regulation of tissue factor activity in endothelium. J Exp Med. 1995; 182(6):1807-1814.
56, Foltyn Zadura A, Memon A A, Stojanovich L, et al. Factor H Autoantibodies in Patients with Antiphospholipid Syndrome and Thrombosis. J Rheumatol. 2015; 42(10):1786-1793.
57. Foltyn Zadura A, Zipfel P F, Bokarewa M I, et al. Factor H autoantibodies and deletion of Complement Factor H-Related protein-1 in rheumatic diseases in comparison to atypical hemolytic uremic syndrome. Arthritis Res Ther. 2012; 14(4):R185.
58. Nakamura H, Oku K, Ogata Y, et al. Alternative pathway activation due to low level of complement factor H in primary antiphospholipid syndrome. Thromb Res. 2018; 164:63-68.
59. Gropp K, Weber N, Reuter M, et al. beta(2)-glycoprotein the major target in antiphospholipid syndrome, is a special human complement regulator. Blood. 2011; 118(10):2774-2783.
60. Arfors L, Lefvert A K. Enrichment of antibodies against phospholipids in circulating immune complexes (CIC) in the anti-phospholipid syndrome (APLS). Clin Exp Immunol. 1997; 108(1):47-51.
61. Noris M, Caprioli J, Bresin E, et al. Relative role of genetic complement abnormalities in sporadic and familial aHUS and their impact on clinical phenotype. Clin J Am Soc Nephrol. 2010; 5(10):1844-1859.
62. Appenzeller S, Souza F H, Wagner Silva de Souza A, Shoenfeld Y, de Carvalho J F. HELLP syndrome and its relationship with antiphospholipid syndrome and antiphospholipid antibodies. Semin Arthritis Rheum. 2011; 41(3):517-523.
63, Rodriguez de Cordoba S. aHUS: a disorder with many risk factors. Blood. 2010; 115(2):158-160.

1. A method for identifying a patient having a complement-mediated disease as likely to benefit from treatment with a Factor D inhibitor comprising the steps of:
(a) in a first assay, (i) incubating serum obtained from the patient with a plurality of glycosylphosphatidylinositol-anchored protein (GPI-AP) deficient cells; (ii) staining the cells with an anti-β5b9 monoclonal antibody (mAb); and (iii) performing flow cytometry to measure C5b9 deposition on the cell membrane;
(b) in a second assay, (i) incubating serum obtained from the patient with a plurality of GPI-AP deficient cells; (ii) adding a terminal complement inhibitor; (iii) staining the cells with an anti-β5b9 mAb; and (iv) performing flow cytometry to measure C5b9 deposition on the cell membrane; and
(c) in a third assay, (i) incubating serum obtained from the patient with a plurality of GPI-AP deficient cells; (ii) adding the Factor D inhibitor; (iii) staining the cells with an anti-C5b9 mAb; and (iv) performing flow cytometry to measure C5b9 deposition on the cell membrane,
wherein the patient is likely to benefit from treatment with the Factor D inhibitor if C5b9 deposition is blocked in the assays of steps (b) and (c), and wherein the patient is not likely to benefit from treatment with the Factor D inhibitor if C5b9 deposition is blocked in the assay of step (b) and not blocked in the assay of step (c).

2. The method of claim 1, wherein the plurality of GPI-AP deficient cells is biochemically treated to remove GPI-AP.

3. The method of claim 1, wherein the plurality of GPI-AP deficient cells is a phosphatidylinositol glycan class A (PIGA) null mutant cell line.

4. The method of claim 1, wherein the terminal complement inhibitor comprises a C5 inhibitor.

5. The method of claim 4, wherein the C5 inhibitor comprises eculizumab, ravulizumab, coversin, cemdisiran, LFG-316, SOBI005, SKY59, REGN3918, ABP959, GNR-45, zimura, RA101495, ISU305, or mubodina.

6. The method of claim 1, wherein the terminal complement inhibitor comprises an anti-β5 mAb.

7. The method of claim 6, wherein the anti-β5 mAb comprises eculizumab or ravulizumab.

8. The method of claim 1, wherein the Factor D inhibitor comprises ACH-4471, ACH-5528 or ACHI-5548.

9. A method for identifying a patient having a complement-mediated disease as likely to benefit from treatment with an inhibitor of the alternative pathway of complement comprising the steps of:
(a) in a first assay, (i) incubating serum obtained from the patient with a plurality of glycosylphosphatidylinositol-anchored protein (GPI-AP) deficient cells; (ii) staining the cells with an anti-β5b9 monoclonal antibody (mAb); and (iii) performing flow cytometry to measure C5b9 deposition on the cell membrane;
(b) in a second assay, (i) incubating serum obtained from the patient with a plurality of GPI-AP deficient cells; (ii) adding a terminal complement inhibitor; (iii) staining the cells with an anti-β5b9 mAb; and (iv) performing flow cytometry to measure C5b9 deposition on the cell membrane; and
(c) in a third assay, (i) incubating serum obtained from the patient with a plurality of GPI-AP deficient cells; (ii) adding the alternative pathway of complement inhibitor; (iii) staining the cells with an anti-β5b9 mAb; and (iv) performing flow cytometry to measure C5b9 deposition on the cell membrane,
wherein the patient is likely to benefit from treatment with the alternative pathway of complement inhibitor if C5b9 deposition is blocked in the assays of steps (b) and (c), and wherein the patient is not likely to benefit from treatment with the alternative pathway of complement inhibitor if C5b9 deposition is blocked in the assay of step (b) and not blocked in the assay of step (c).

10. The method of claim 9, wherein the alternative pathway of complement inhibitor comprises a Factor B inhibitor.

11. The method of claim 10, wherein the Factor B inhibitor comprises IONIS-FB-LRx.

12. The method of claim 9, wherein the plurality of GPI-AP deficient cells is biochemically treated to remove GPI-AP.

13. The method of claim 9, wherein the plurality of GPI-AP deficient cells is a phosphatidylinositol glycan class A (PIGA) null mutant cell line.

14. The method of claim 9, wherein the terminal complement inhibitor comprises a C5 inhibitor.

15. The method of claim 14, wherein the C5 inhibitor comprises eculizumab, ravulizumab, coversin, cemdisiran, LFG-316, SOBI005, SKY59, REGN3918, ABP959, GNR-45, zimura, RA101495, ISU305, or mubodina.

16. The method of claim 9, wherein the terminal complement inhibitor comprises an anti-β5 mAb.

17. The method of claim 16, wherein the anti-CS mAb comprises eculizumab or ravulizumab.

18. A method of treating a patient having a complement-mediated disease comprising the step of administering a Factor D inhibitor to the patient identified using the method of claim 1.

19. A method of treating a patient having a complement-mediated disease comprising the step of administering an alternative pathway of complement inhibitor to the patient identified using the method of claim 9.
